(19) United States
(12) Patent Application Publication
(10) Pub. No.: US 2021/0332418 A1
(43) Pub. Date: Oct. 28, 2021

(54) SYSTEMS AND METHODS FOR MICROBIOME ANALYSIS

(71) Applicant: Viome, Inc., Bellevue, WA (US)

(72) Inventors: Momchilo VUYISICH, Bothell, WA (US); Ally PERLINA, San Diego, CA (US); Guruduth S. BANAVAR, Pelham Manor, NY (US); Helen MESSIER, Cupertino, CA (US)

(21) Appl. No.: 17/273,463

(22) PCT Filed: Sep. 6, 2019
(86) PCT No.: PCT/US19/50102

§ 371 Date: Mar. 4, 2021

Related U.S. Application Data
(60) Provisional application No. 62/728,035, filed on Sep. 6, 2018.

(51) Int. Cl. C12Q 1/689; G16H 10/40; G16H 20/10; G16H 20/60; G16H 50/30

(52) U.S. Cl. CPC C12Q 1/689; G16H 10/40; G16H 50/30; G16H 20/60; G16H 20/10

(57) ABSTRACT

Provided are methods balancing a gut microbiome. Methods involve sequencing nucleic acids from a sample to determine quantifies or activities of microorganisms Sequence information is used to generate functional activity scores for functional categories. These scores can be actionable. Methods also include generating recommendations involving administration of food, supplements, or medication to affect quantities or activities of microorganisms

### FIELD

The present disclosure relates to methods for identifying and quantifying microorganisms in a heterogeneous sample using sequencing data. Disclosed further are methods of generating recommendations to affect the growth or activity of these microorganisms.

### BACKGROUND

All plants and animals, from simple life forms to humans, live in close association with microbial organisms. Microbiota have been found to be crucial for immunologic, hormonal, and metabolic homeostasis of their host. The ability to identify, quantify, and affect the microbiome provides an opportunity to influence relative abundancies of microorganisms as well as their activity in a host, thereby affecting the health status of the host. Use of next generation sequencing (NGS) is becoming a common method to monitor these microbial systems. However, methods to accurately identify the specific microorganisms contained in a sample, as well as their relative abundance, in a reliable and time efficient manner, have remained a challenge. Further, application of this information to generate a recommendation to affect the health status of the host/subject has not yet been accomplished.

### SUMMARY

Provided herein is a method comprising: a) generating a dataset about a subject by: i) determining data from a gut microbiome from a subject by: (1) obtaining nucleic acids from a sample comprising gut microbiome from a subject; (2) obtaining sequence information representative of the nucleic acids; ii) determining from the sequence information, (A) quantitative measures of each of a plurality of different microbial taxa and (B) quantitative measures of activity of each of a plurality of different genes; b) determining from the dataset a functional activity score for at least one functional category; c) determining that the functional activity score is outside a reference range; and d) recommending alterations in a diet for, or altering a diet of, the subject to alter the gut microbiome, wherein altering the gut microbiome shifts the functional score toward or into the reference range. In one embodiment the functional category includes one or more biochemical pathway components, one or more taxa group components or a combination of at least one biochemical pathway component and at least one taxa group component. In another embodiment the functional category is an aggregate functional category comprising a plurality of functional subcategories, each subcategory including at least one biochemical pathway component and at least one taxa group component. In another embodiment the quantitative measures of activity the plurality of genes are used to determine a quantitative measure of activity of a biochemical pathway. In another embodiment the quantitative measures of the plurality of microbial taxa are used to determine a quantitative measure of activity of a taxa group. In another embodiment the method comprises, before operation (b): determining from the dataset a biochemical pathway activity component and/or a taxa group activity component. In another embodiment the nucleic acids comprise ribonucleic acids. In another embodiment the ribonucleic acids comprise RNA. In another embodiment the ribonucleic acids comprise mRNA. In another embodiment the ribonucleic acids are essentially free of non-informative RNA. In another embodiment the quantitative measures are absolute measures or relative measures. In another embodiment at least one functional category is selected from the group consisting of inflammatory activity, metabolic fitness, digestive efficiency, intestinal barrier health, protein fermentation, gas production, microbial richness, butyrate production pathways, LPS biosynthesis pathways, methane gas production pathways, sulfide gas production pathways, flagellar assembly pathways, ammonia production pathways, putrescine production pathways, oxalate metabolism pathways, uric acid production pathways, salt stress pathways, biofilm chemotaxis and virulence pathways, TMA production pathways, primary bile acid pathways, secondary bile acid pathways, salt stress pathways, fucose metabolism pathways, cadaverine production pathways. In another embodiment a reference range is determined from a plurality of control subjects, wherein the plurality is at least any of 100 subjects, 1000 subjects, 5000, subjects or 10,000 subjects. In another embodiment an activity score outside of reference range is above or below the reference range. In another embodiment alterations in the diet comprise one or more of (1) adding to the diet microbes of one or more taxa that produce desired nutrients; (2) adding to the diet one or more dietary items that promote activity of microbes of one or more taxa to produce desired nutrients; (3) adding to the diet one or more dietary items that suppress the production by microbes of undesired compounds. In another embodiment alterations in the diet comprise one or more of (1) adding to the diet microbes of one or more taxa for which there is a deficiency in the gut of the subject; (2) adding to the diet food or nutrients that promote growth of microbes of one or more taxa for which there is a deficiency; (3) adding to the diet a food or supplement comprising a nutrient that is deficient in the diet; and (4) adding to the diet a food or supplement comprising a nutrient that suppresses growth of microbes of one or more taxa for which there is an excess in the gut of the subject. In another embodiment recommending comprises transmitting recommendations to the subject over an electronic communications network. In another embodiment transmitting comprises posting recommendations to a website, e.g., a password-protected website, that is accessible to the subject.

In another aspect provided herein is a method comprising: a) determining for a subject a functional activity score for at least one functional category, where in the functional activity score is derived from a dataset comprising nucleic acid sequence-derived quantitative measures of each of a plurality of different microbial taxa and quantitative measures of activity of each of a plurality of different genes; b) determining that the functional activity score is outside a reference range; and c) transmitting to the subject a dietary item determined, when included in the diet of the subject, to alter the gut microbiome so as to shift the functional score toward or into the reference range. In one embodiment, the method comprises, before operation (a), receiving a gut microbiome sample from the subject and sequencing microbial nucleic acids in the sample to produce nucleic acid sequences.

In another aspect provided herein is a method comprising: (a) receiving from the subject a sample comprising microbes from the subject's microbiome; (b) sequencing a transcriptome from the microbiome; (c) determining, from the sequence transcriptome, a functional utility score for each of one or more functional categories; (d) determining that the functional utility score is outside a reference range; (e) generating a dietary model including one or more dietary items which, when included in a diet of the subject, will move functional score toward or into the reference range; and (f) providing the dietary model or at least one of the dietary items to the subject.

Provided herein is a method of improving a health characteristic in a subject comprising: obtaining nucleic acids from a sample from the subject; obtaining sequence information representative of the nucleic acids; assigning a taxonomic designation to a portion of the nucleic acids having sequence signatures informative of the taxonomic designation; quantifying an amount of microorganisms in the sample corresponding to the taxonomic designation; comparing the quantity of microorganisms in the sample corresponding to the taxonomic designation to a reference value for the microorganism corresponding to the taxonomic designation; identifying a nutrient that alters the growth of microorganisms corresponding to the taxonomic designation; and recommending or administering to the subject a food comprising the nutrient that alters the growth of microorganisms corresponding to the taxonomic designation. Further provided herein are methods wherein quantifying an amount of microorganisms comprises quantifying relative amounts of a plurality of microorganisms. Further provided herein are methods wherein administration of the food adjusts the relative amounts of microorganisms in the subject to approach the reference value. Further provided herein are methods wherein the reference value represents a benchmark for the health characteristic. Further provided herein are methods wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. Further provided herein are methods wherein the sample comprises feces. Further provided herein are methods wherein the health characteristic comprises an immune system function, an endocrine system function, a digestive system function, a metabolic function, a mental health characteristic, or any combination thereof. Further provided herein are methods wherein the immune system function comprises an immune tolerance, an autoimmune disease, an inflammation, an infection, or any combination thereof. Further provided herein are methods wherein the autoimmune disease comprises celiac disease, diabetes mellitus type 1, Graves' disease, inflammatory bowel disease, multiple sclerosis, psoriasis, rheumatoid arthritis, systemic lupus erythematosus, or any combination thereof. Further provided herein are methods wherein the infection comprises a respiratory infection, a urinary tract infection, a Clostridium difficile infection, or any combination thereof. Further provided herein are methods wherein the endocrine system function comprises a thyroid function, a hormone level, a cholesterol level change, or any combination thereof. Further provided herein are methods wherein the hormone level is a cortisol level, an estrogen level, an insulin level, a leptin level, a melatonin level, an oxytocin level, a testosterone level, or any combination thereof. Further provided herein are methods wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. Further provided herein are methods wherein the metabolic function comprises glucose metabolism, weight gain, weight loss, insulin level, fat mass accumulation, or any combination thereof. Further provided herein are methods wherein the mental health characteristic comprises anxiety, depression, Parkinson's disease, autism spectrum disorder, or a combination thereof. Further provided herein are methods wherein the nucleic acids obtained comprise RNA, and wherein the RNA comprises mRNA, tRNA, rRNA, snRNA, or any combination thereof. Further provided herein are methods wherein the nucleic acids obtained comprise DNA. Further provided herein are methods wherein the food comprises a micronutrient. Further provided herein are methods wherein the micronutrient increases growth of a microbial taxon. Further provided herein are methods wherein the micronutrient increases a quantity of a microbial taxon. Further provided herein are methods wherein the micronutrient suppresses growth of a microbial taxon. Further provided herein are methods, wherein the micronutrient decreases a quantity of a microbial taxon. Further provided herein are methods wherein the micronutrient increases a quantity of a first microbial taxon that competes with a second microbial taxon. Further provided herein are methods wherein the micronutrient increases a quantity of a first microbial taxon that increases a quantity of a second microbial taxon. Further provided herein are methods wherein the micronutrient increases the bioavailability of a substance that improves health. Further provided herein are methods wherein the micronutrient increases the uptake of a substance that improves health. Further provided herein are methods wherein the micronutrient comprises a mineral, a trace mineral, a vitamin, a biochemical substrate, or any combination thereof. Further provided herein are methods wherein the mineral is calcium, magnesium, sulfur, or any combination thereof. Further provided herein are methods wherein the trace mineral is iron, chromium, copper, fluoride, iodine, manganese, molybdenum, selenium, zinc, or any combination thereof. Further provided herein are methods wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof

Provided herein are methods of improving a health characteristic in a subject comprising: obtaining nucleic acids from a sample from the subject; obtaining sequence information representative of the nucleic acids; assigning a taxonomic designation to a portion of the nucleic acids having sequence signatures informative of the taxonomic designation; quantifying an amount of microorganisms in the sample corresponding to the taxonomic designation; comparing the quantity of microorganisms in the sample corresponding to the taxonomic designation to a reference value for the microorganism corresponding to the taxonomic designation; identifying a nutrient that alters the growth of microorganisms corresponding to the taxonomic designation; and recommending or administering to the subject a supplement comprising the nutrient that alters the growth of microorganisms corresponding to the taxonomic designation. Further provided herein are methods wherein quantifying an amount of microorganisms comprises quantifying relative amounts of a plurality of microorganisms. Further provided herein are methods wherein administration of the supplement adjusts the relative amounts of microorganisms in the subject to approach the reference value. Further provided herein are methods wherein the reference value represents a benchmark for the health characteristic. Further provided herein are methods wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. Further provided herein are methods wherein the sample comprises feces. Further provided herein are methods wherein the health characteristic comprises an immune system function, an endocrine system function, a digestive system function, a metabolic function, a mental health characteristic, or any combination thereof. Further provided herein are methods wherein the immune system function comprises an immuno-tolerance, an autoimmune disease, an inflammation, an infection, or any combination thereof. Further provided herein are methods wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. Further provided herein are methods wherein the infection comprises a respiratory infection, a urinary tract infection, a Clostridium difficile infection, or any combination thereof. Further provided herein are methods wherein the endocrine system function comprises a thyroid function, a hormone level, a cholesterol level change, or any combination thereof. Further provided herein are methods wherein the hormone level is a cortisol level, an estrogen level, an insulin level, a leptin level, a melatonin level, an oxytocin level, a testosterone level, or any combination thereof. Further provided herein are methods wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. Further provided herein are methods wherein the metabolic function comprises glucose metabolism, weight gain, weight loss, insulin level, fat mass accumulation, or any combination thereof. Further provided herein are methods wherein the mental health characteristic comprises anxiety, depression, Parkinson's disease, autism spectrum disorder, or a combination thereof. Further provided herein are methods wherein the nucleic acids obtained comprise RNA, and wherein the RNA comprises mRNA, tRNA, rRNA, snRNA, or any combination thereof. Further provided herein are methods wherein the nucleic acids obtained comprise DNA. Further provided herein are methods wherein the supplement comprises a micronutrient. Further provided herein are methods wherein the micronutrient increases growth of a microbial taxon. Further provided herein are methods wherein the micronutrient increases a quantity of a microbial taxon. Further provided herein are methods herein the micronutrient suppresses growth of a microbial taxon. Further provided herein are methods wherein the micronutrient decreases a quantity of a microbial taxon. Further provided herein are methods wherein the micronutrient increases a quantity of a first microbial taxon that competes with a second microbial taxon. Further provided herein are methods wherein the micronutrient increases a quantity of a first microbial taxon that increases a quantity of a second microbial taxon. Further provided herein are methods wherein the micronutrient increases the bioavailability of a substance that improves health. Further provided herein are methods wherein the micronutrient increases the uptake of a substance that improves health. Further provided herein are methods wherein the micronutrient comprises a mineral, a trace mineral, a vitamin, a biochemical substrate, or any combination thereof. Further provided herein are methods wherein the mineral is calcium, magnesium, sulfur, or any combination thereof. Further provided herein are methods wherein the trace mineral is iron, chromium, copper, fluoride, iodine, manganese, molybdenum, selenium, zinc, or any combination thereof. Further provided herein are methods wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof. Further provided herein are methods wherein the supplement comprises a prebiotic material, magnesium, fish oil, or any combination thereof

Provided herein are methods of improving a health characteristic in a subject comprising: obtaining nucleic acids from a sample from the subject; obtaining sequence information representative of the nucleic acids; assigning a taxonomic designation to a portion of the nucleic acids having sequence signatures informative of the taxonomic designation; quantifying an amount of microorganisms in the sample corresponding to the taxonomic designation; comparing the quantity of microorganisms in the sample corresponding to the taxonomic designation to a reference value for the microorganism corresponding to the taxonomic designation; identifying a medication that alters the growth of microorganisms corresponding to the taxonomic designation; and recommending or administering to the subject the medication that alters the growth of microorganisms corresponding to the taxonomic designation. Further provided herein are methods wherein quantifying an amount of microorganisms comprises quantifying relative amounts of a plurality of microorganisms. Further provided herein are methods wherein administration of the medication adjusts the relative amounts of microorganisms in the subject to approach the reference value. Further provided herein are methods wherein the reference value represents a benchmark for the health characteristic. Further provided herein are methods wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. Further provided herein are methods wherein the sample comprises feces. Further provided herein are methods wherein the health characteristic comprises an immune system function, an endocrine system function, a digestive system function, a metabolic function, a mental health characteristic, or any combination thereof. Further provided herein are methods wherein the immune system function comprises an immune tolerance, an autoimmune disease, an inflammation, an infection, or any combination thereof. Further provided herein are methods wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. Further provided herein are methods wherein the infection comprises a respiratory infection, a urinary tract infection, a Clostridium difficile infection, or any combination thereof. Further provided herein are methods wherein the endocrine system function comprises a thyroid function, a hormone level, a cholesterol level change, or any combination thereof. Further provided herein are methods wherein the hormone level is a cortisol level, an estrogen level, an insulin level, a leptin level, a melatonin level, an oxytocin level, a testosterone level, or any combination thereof. Further provided herein are methods wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. Further provided herein are methods wherein the metabolic function comprises glucose metabolism, weight gain, weight loss, insulin level, fat mass accumulation, or any combination thereof. Further provided herein are methods wherein the mental health characteristic comprises anxiety, depression, Parkinson's disease, autism spectrum disorder, or a combination thereof. Further provided herein are methods wherein the nucleic acids obtained comprise RNA, and wherein the RNA comprises mRNA, tRNA, rRNA, snRNA, or any combination thereof. Further provided herein are methods wherein the nucleic acids obtained comprise DNA. Further provided herein are methods wherein the medication suppresses growth of microorganisms corresponding to the taxonomic designation. Further provided herein are methods wherein the medication is an antibiotic, an antiviral, an antipsychotic, an acid-reducing medication, a chemotherapy drug, or a blood-pressure medication. Further provided herein are methods wherein the antibiotic is selected from a penicillin, a cephalosporin, a macrolide, a fluoroquinolone, a sulfonamide, a tetracycline, or an aminoglycoside. Further provided herein are methods wherein the medication supports growth of microorganisms corresponding to the taxonomic designation.

Provided herein are methods of improving a health characteristic in a subject comprising: obtaining nucleic acids from a sample from the subject; obtaining sequence information representative of the nucleic acids; quantifying an amount of the nucleic acids in the sample corresponding to a biochemical activity; comparing the quantity of the biochemical activity in the sample to a reference value for the biochemical activity; identifying a nutrient that alters the amount of the biochemical activity in a gut of the subject; and recommending or administering to the subject a food containing the nutrient that alters the amount of the biochemical activity in the gut of the subject. Further provided herein are methods wherein administration of the food adjusts the biochemical activity in the subject to approach the reference value. Further provided herein are methods wherein the reference value represents a benchmark for the health characteristic. Further provided herein are methods wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. Further provided herein are methods wherein the sample comprises feces. Further provided herein are methods wherein the health characteristic comprises an immune system function, an endocrine system function, a digestive system function, a metabolic function, a mental health characteristic, or any combination thereof. Further provided herein are methods wherein the immune system function comprises an immune tolerance, an autoimmune disease, an inflammation, an infection, or any combination thereof. Further provided herein are methods wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. Further provided herein are methods wherein the infection comprises a respiratory infection, a urinary tract infection, a Clostridium difficile infection, or any combination thereof. Further provided herein are methods wherein the endocrine system function comprises a thyroid function, a hormone level, a cholesterol level change, or any combination thereof. Further provided herein are methods wherein the hormone level is a cortisol level, an estrogen level, an insulin level, a leptin level, a melatonin level, an oxytocin level, a testosterone level, or any combination thereof. Further provided herein are methods wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. Further provided herein are methods wherein the metabolic function comprises glucose metabolism, weight gain, weight loss, insulin level, fat mass accumulation, or any combination thereof. Further provided herein are methods wherein the mental health characteristic comprises anxiety, depression, Parkinson's disease, autism spectrum disorder, or a combination thereof. Further provided herein are methods wherein the nucleic acids obtained comprise RNA, and wherein the RNA comprises mRNA, tRNA, rRNA, snRNA, or any combination thereof. Further provided herein are methods wherein the nucleic acids obtained comprise DNA. Further provided herein are methods further comprising quantifying a small molecule informative of an amount of the biochemical activity. Further provided herein are methods wherein the quantifying comprises an enzymatic activity assay, a growth-inhibition culture, metabolic profiling, or any combination thereof. Further provided herein are methods wherein the small molecule comprises a metabolite generated by the biochemical activity. Further provided herein are methods wherein the small molecule comprises a short-chain fatty acid. Further provided herein are methods wherein the short-chain fatty acid comprises a butyrate. Further provided herein are methods wherein the small molecule comprises a propionate. Further provided herein are methods wherein the small molecule comprises a substrate of the biochemical activity. Further provided herein are methods wherein the food comprises a micronutrient. Further provided herein are methods wherein the micronutrient increases growth of a microbial taxon that generates the biochemical activity. Further provided herein are methods wherein the micronutrient increases a quantity of a microbial taxon that generates the biochemical activity. Further provided herein are methods wherein the micronutrient increases a level of the biochemical activity in the gut of the subject. Further provided herein are methods wherein the micronutrient suppresses growth of a microbial taxon that generates the biochemical activity. Further provided herein are methods wherein the micronutrient decreases a quantity of a microbial taxon that generates the biochemical activity. Further provided herein are methods wherein the micronutrient decreases the level of the biochemical activity in the gut of the subject. Further provided herein are methods wherein the micronutrient supplements the biochemical activity. Further provided herein are methods wherein the micronutrient activates the biochemical activity. Further provided herein are methods wherein the micronutrient inhibits the biochemical activity. Further provided herein are methods wherein the micronutrient increases a quantity of a first microbial taxon that competes with a second microbial taxon, wherein the second microbial taxon generates the biochemical activity. Further provided herein are methods wherein the micronutrient increases a quantity of a first microbial taxon that increases a quantity of a second microbial taxon, wherein the second microbial taxon generates the biochemical activity. Further provided herein are methods wherein the micronutrient increases the bioavailability of a substance that improves health. Further provided herein are methods wherein the micronutrient increases the uptake of a substance that improves health. Further provided herein are methods wherein the micronutrient comprises a mineral, a trace mineral, a vitamin, a biochemical substrate, or any combination thereof. Further provided herein are methods wherein the mineral is calcium, magnesium, sulfur, or any combination thereof. Further provided herein are methods wherein the trace mineral is iron, chromium, copper, fluoride, iodine, manganese, molybdenum, selenium, zinc, or any combination thereof. Further provided herein are methods wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof. Further provided herein are methods further comprising: assigning a taxonomic designation to a portion of the nucleic acids having sequence signatures informative of the taxonomic designation; assigning the biochemical activity to the taxonomic designation; quantifying an amount of microorganisms in the sample corresponding to the taxonomic designation; and comparing the quantity of microorganisms in the sample corresponding to the taxonomic designation to a reference value for the microorganism corresponding to the taxonomic designation.

Provided herein are methods of improving a health characteristic in a subject comprising: obtaining nucleic acids from a sample from the subject; obtaining sequence information representative of the nucleic acids; quantifying an amount of the nucleic acids in the sample corresponding to a biochemical activity; comparing the quantity of the biochemical activity in the sample to a reference value for the biochemical activity; identifying a nutrient that alters the amount of the biochemical activity in a gut of the subject; and recommending or administering to the subject a supplement containing the nutrient that alters the amount of the biochemical activity in the gut of the subject. Further provided herein are methods wherein administration of the supplement adjusts the biochemical activity in the subject to approach the reference value. Further provided herein are methods wherein the reference value represents a benchmark for the health characteristic. 128. The method of embodiment 125, wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. Further provided herein are methods wherein the sample comprises feces. Further provided herein are methods wherein the health characteristic comprises an immune system function, an endocrine system function, a digestive system function, a metabolic function, a mental health characteristic, or any combination thereof. Further provided herein are methods wherein the immune system function comprises an immune tolerance, an autoimmune disease, an inflammation, an infection, or any combination thereof. Further provided herein are methods wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. Further provided herein are methods wherein the infection comprises a respiratory infection, a urinary tract infection, a Clostridium difficile infection, or any combination thereof. Further provided herein are methods wherein the endocrine system function comprises a thyroid function, a hormone level, a cholesterol level change, or any combination thereof. Further provided herein are methods wherein the hormone level is a cortisol level, an estrogen level, an insulin level, a leptin level, a melatonin level, an oxytocin level, a testosterone level, or any combination thereof. Further provided herein are methods wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. Further provided herein are methods wherein the metabolic function comprises glucose metabolism, weight gain, weight loss, insulin level, fat mass accumulation, or any combination thereof. Further provided herein are methods wherein the mental health characteristic comprises anxiety, depression, Parkinson's disease, autism spectrum disorder, or a combination thereof. Further provided herein are methods wherein the nucleic acids obtained comprise RNA, and wherein the RNA comprises mRNA, tRNA, rRNA, snRNA, or any combination thereof. Further provided herein are methods wherein the nucleic acids obtained comprise DNA. Further provided herein are methods further comprising quantifying a small molecule informative of an amount of the biochemical activity. Further provided herein are methods wherein the quantifying comprises an enzymatic activity assay, a growth-inhibition culture, metabolic profiling, or any combination thereof. Further provided herein are methods wherein the small molecule comprises a metabolite generated by the biochemical activity. Further provided herein are methods wherein the small molecule comprises a short-chain fatty acid. Further provided herein are methods wherein the short-chain fatty acid comprises a butyrate. Further provided herein are methods wherein the small molecule comprises a propionate. Further provided herein are methods wherein the small molecule comprises a substrate of the biochemical activity. Further provided herein are methods wherein the supplement comprises a micronutrient. Further provided herein are methods wherein the micronutrient increases growth of a microbial taxon that generates the biochemical activity. Further provided herein are methods wherein the micronutrient increases a quantity of a microbial taxon that generates the biochemical activity. Further provided herein are methods wherein the micronutrient increases a level of the biochemical activity in the gut of the subject. Further provided herein are methods wherein the micronutrient suppresses growth of a microbial taxon that generates the biochemical activity. Further provided herein are methods wherein the micronutrient decreases a quantity of a microbial taxon that generates the biochemical activity. Further provided herein are methods wherein the micronutrient decreases the level of the biochemical activity in the gut of the subject. Further provided herein are methods wherein the micronutrient supplements the biochemical activity. Further provided herein are methods wherein the micronutrient activates the biochemical activity. Further provided herein are methods wherein the micronutrient inhibits the biochemical activity. Further provided herein are methods wherein the micronutrient increases a quantity of a first microbial taxon that competes with a second microbial taxon, wherein the second microbial taxon generates the biochemical activity. 1 Further provided herein are methods wherein the micronutrient increases a quantity of a first microbial taxon that increases a quantity of a second microbial taxon, wherein the second microbial taxon generates the biochemical activity. Further provided herein are methods wherein the micronutrient increases the bioavailability of a substance that improves health. Further provided herein are methods wherein the micronutrient increases the uptake of a substance that improves health. Further provided herein are methods wherein the supplement comprises a mineral, a trace mineral, a vitamin, a biochemical substrate, or any combination thereof. Further provided herein are methods wherein the mineral is calcium, magnesium, sulfur, or any combination thereof. Further provided herein are methods wherein the trace mineral is iron, chromium, copper, fluoride, iodine, manganese, molybdenum, selenium, zinc, or any combination thereof. Further provided herein are methods wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof. Further provided herein are methods wherein the supplement comprises a prebiotic material, magnesium, fish oil, or any combination thereof. Further provided herein are methods further comprising: assigning a taxonomic designation to a portion of the nucleic acids having sequence signatures informative of the taxonomic designation; assigning the biochemical activity to the taxonomic designation; quantifying an amount of microorganisms in the sample corresponding to the taxonomic designation; and comparing the quantity of microorganisms in the sample corresponding to the taxonomic designation to a reference value for the microorganism corresponding to the taxonomic designation.

Provided herein are methods of improving a health characteristic in a subject comprising: obtaining nucleic acids from a sample from the subject; obtaining sequence information representative of the nucleic acids; quantifying an amount of the nucleic acids in the sample corresponding to a biochemical activity; comparing the quantity of the biochemical activity in the sample to a reference value for the biochemical activity; identifying a nutrient that alters the amount of the biochemical activity in a gut of the subject; and recommending or administering to the subject a medication that alters the amount of the biochemical activity in the gut of the subject. Further provided herein are methods wherein administration of the medication adjusts the biochemical activity in the subject to approach the reference value. Further provided herein are methods wherein the reference value represents a benchmark for the health characteristic. Further provided herein are methods wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. Further provided herein are methods wherein the sample comprises feces. Further provided herein are methods wherein the health characteristic comprises an immune system function, an endocrine system function, a digestive system function, a metabolic function, a mental health characteristic, or any combination thereof. Further provided herein are methods wherein the immune system function comprises an immune tolerance, an autoimmune disease, an inflammation, an infection, or any combination thereof. Further provided herein are methods wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. Further provided herein are methods wherein the infection comprises a respiratory infection, a urinary tract infection, a Clostridium difficile infection, or any combination thereof. Further provided herein are methods wherein the endocrine system function comprises a thyroid function, a hormone level, a cholesterol level change, or any combination thereof. Further provided herein are methods wherein the hormone level is a cortisol level, an estrogen level, an insulin level, a leptin level, a melatonin level, an oxytocin level, a testosterone level, or any combination thereof. Further provided herein are methods wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. Further provided herein are methods wherein the metabolic function comprises glucose metabolism, weight gain, weight loss, insulin level, fat mass accumulation, or any combination thereof. Further provided herein are methods wherein the mental health characteristic comprises anxiety, depression, Parkinson's disease, autism spectrum disorder, or a combination thereof. Further provided herein are methods wherein the nucleic acids obtained comprise RNA, and wherein the RNA comprises mRNA, tRNA, rRNA, snRNA, or any combination thereof. Further provided herein are methods wherein the nucleic acids obtained comprise DNA. Further provided herein are methods further comprising quantifying a small molecule informative of an amount of the biochemical activity. Further provided herein are methods wherein the quantifying comprises an enzymatic activity assay, a growth-inhibition culture, metabolic profiling, or any combination thereof. Further provided herein are methods wherein the small molecule comprises a metabolite generated by the biochemical activity. Further provided herein are methods wherein the small molecule comprises a short-chain fatty acid. Further provided herein are methods wherein the short-chain fatty acid comprises a butyrate. Further provided herein are methods wherein the small molecule comprises a propionate. Further provided herein are methods wherein the small molecule comprises a substrate of the biochemical activity. Further provided herein are methods wherein the medication suppresses growth of microorganisms corresponding to the taxonomic designation. Further provided herein are methods wherein the medication is an antibiotic, an antiviral, an antipsychotic, an acid-reducing medication, a chemotherapy drug, or a blood-pressure medication. Further provided herein are methods wherein the antibiotic is selected from a penicillin, a cephalosporin, a macrolide, a fluoroquinolone, a sulfonamide, a tetracycline, or an aminoglycoside. Further provided herein are methods wherein the medication supports growth of microorganisms corresponding to the taxonomic designation. Further provided herein are methods further comprising: assigning a taxonomic designation to a portion of the nucleic acids having sequence signatures informative of the taxonomic designation; assigning the biochemical activity to the taxonomic designation; quantifying an amount of microorganisms in the sample corresponding to the taxonomic designation; and comparing the quantity of microorganisms in the sample corresponding to the taxonomic designation to a reference value for the microorganism corresponding to the taxonomic designation.

Provided herein are methods of taxonomically classifying organisms in a heterogeneous sample comprising: obtaining ribonucleic acids from a heterogeneous sample; obtaining sequence information representative of the ribonucleic acids; assaying for the presence of a plurality of sequence signatures in the sequence information informative of a taxonomic designation; categorizing the sample as comprising an organism corresponding to the taxonomic designation; and identifying a recommendation consistent with the presence of the organism corresponding to the taxonomic designation.

Provided herein are methods of taxonomically assessing the biochemical activity of a heterogeneous sample comprising: obtaining ribonucleic acids from a heterogeneous sample; obtaining sequence information representative of the ribonucleic acids; assigning the ribonucleic acids to open reading frames; identifying biochemical activities in the sample consistent with the open reading frames; and identifying a recommendation consistent with the presence of the biochemical activity.

Provided herein are methods of identifying a health recommendation for a subject comprising: obtaining ribonucleic acids from a heterogeneous sample; obtaining sequence information representative of the ribonucleic acids; assaying for a plurality of sequence signatures in the sample informative of a taxonomic designation; quantifying an amount of an organism corresponding to the taxonomic designation present in the sample; and identifying a health recommendation consistent with the amount of the organism corresponding to the taxonomic designation.

Provided herein are methods of taxonomically assessing the biochemical activity of a heterogeneous sample comprising: obtaining ribonucleic acids from a heterogeneous sample; obtaining sequence information representative of the ribonucleic acids; assigning taxonomic designations to a portion of the ribonucleic acids having sequence signatures informative of a taxonomic designation; assigning the ribonucleic acids to open reading frames consistent with the taxonomic designations; quantifying a biochemical activity in the sample consistent with the open reading frames; and identifying a recommendation consistent with the quantity of the biochemical activity.

Provided herein are methods of assessing gut shedding in a subject comprising: obtaining a nucleic acids from a stool sample; obtaining sequence information representative of the nucleic acids; assaying for a presence of a plurality of sequence signatures in the stool sample informative of a category of organisms; quantifying an amount of nucleic acid derived from the subject in the sample; quantifying an amount of nucleic acid derived from microorganisms in the sample; and assessing gut shedding in the subject by determining the proportion of nucleic acid derived from the subject to the proportion of nucleic acid derived from microorganisms. Further provided herein are methods further comprising generating a health recommendation based on the assessing gut shedding in the subject.

Provided herein are methods of identifying antibiotic resistant microorganisms in a heterogeneous microbial sample from a subject comprising: obtaining a ribonucleic acids from a heterogeneous microbial sample; obtaining sequence information representative of the ribonucleic acids; assaying for a presence of one or more sequence signatures in the heterogeneous microbial sample informative of a microorganism that is resistant to an antibiotic; and providing an effective amount of a different antibiotic to the subject to slow growth of the microorganism.

Provided herein are methods of treating a bacterial infection in a subject comprising: obtaining a ribonucleic acids from a heterogeneous microbial sample from the subject; obtaining sequence information representative of the ribonucleic acids; assaying for a presence of a plurality of sequence signatures in the heterogeneous microbial sample informative of a microorganism that is sensitive to an antibiotic; and providing an effective amount of the antibiotic to the subject to slow growth of the microorganism.

Provided herein are methods of assessing a metabolism in a subject comprising: assaying for nucleic acids corresponding to taxa in a gut microbiome of the subject that enhance metabolism; and assaying for nucleic acids corresponding to taxa in the gut microbiome of the subject that reduce metabolism.

Provided herein are methods of assessing metabolism in a subject comprising: assaying for nucleic acids corresponding to taxa in a gut microbiome of the subject that enhance metabolism; and assaying for nucleic acids corresponding to taxa in the gut microbiome of the subject that reduce metabolism; and assessing metabolic activity in the subject in the subject based on the taxa in a gut microbiome of the subject that enhance metabolism and the taxa in the gut microbiome of the subject that reduce metabolism in the gut microbiome. Further provided herein are methods further comprising administering to the subject a probiotic that modulates metabolism-relevant taxa in the gut microbiome. Further provided herein are methods further comprising recommending to the subject a diet that modulates metabolism-relevant taxa in the subject's gut.

Provided herein are methods comprising: a) generating a dataset about a subject by: i) determining data from a gut microbiome from a subject by: (1) obtaining nucleic acids from a sample comprising gut microbiome from a subject; (2) obtaining sequence information representative of the nucleic acids; iii) determining from the sequence information, (A) quantitative measures of each of a plurality of different microbial taxa and (B) quantitative measures of activity of each of a plurality of different genes; ii) determining phenotypic data about a plurality of different phenotypic traits of the subject; b) determining from the dataset a score set comprising a plurality of scores selected from: (i) a metabolic fitness score, (ii) an inflammatory activity score, (iii) a gut lining shedding score, (iv) a microbial richness score and (v) a microbial diversity score; c) recommending or administering to the subject one or more of a food to improve one or more of the scores. Further provided herein are methods wherein recommended foods are partial rank ordered into a plurality of groups from more desirable to less desirable to improve the one or more scores. Further provided herein are methods wherein the plurality of groups is 2, 3, 4 or 5.

Provided herein are methods comprising: a) generating a dataset about a subject by: i) determining data from a gut microbiome from a subject by: (1) obtaining nucleic acids from a sample comprising gut microbiome from a subject; (2) obtaining sequence information representative of the nucleic acids; iii) determining from the sequence information, (A) quantitative measures of each of a plurality of different microbial taxa and (B) quantitative measures of activity of each of a plurality of different genes; ii) determining phenotypic data about a plurality of different phenotypic traits of the subject; b) determining from the dataset a score set comprising a plurality of scores selected from: (i) a metabolic fitness score, (ii) an inflammatory activity score, (iii) a gut lining shedding score, (iv) a microbial richness score and (v) a microbial diversity score; c) recommending or administering to the subject one or more of a supplement to improve one or more of the scores. Further provided herein are methods wherein recommended supplements are partial rank ordered into a plurality of groups from more desirable to less desirable to improve the one or more scores. Further provided herein are methods wherein the plurality of groups is 2, 3, 4 or 5.

Provided herein are methods comprising: a) generating a dataset about a subject by: i) determining data from a gut microbiome from a subject by: (1) obtaining nucleic acids from a sample comprising gut microbiome from a subject; (2) obtaining sequence information representative of the nucleic acids; iii) determining from the sequence information, (A) quantitative measures of each of a plurality of different microbial taxa and (B) quantitative measures of activity of each of a plurality of different genes; ii) determining phenotypic data about a plurality of different phenotypic traits of the subject; b) determining from the dataset a score set comprising a plurality of scores selected from: (i) a metabolic fitness score, (ii) an inflammatory activity score, (iii) a gut lining shedding score, (iv) a microbial richness score and (v) a microbial diversity score; c) recommending or administering to the subject one or more of a medication to improve one or more of the scores. Further provided herein are methods wherein recommended medications are partial rank ordered into a plurality of groups from more desirable to less desirable to improve the one or more scores. Further provided herein are methods wherein the plurality of groups is 2, 3, 4 or 5.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The following references may be relevant to the present disclosure: WO 2019/113563; WO 2018/237209; WO 2019/099574; WO 2018/160899; PCT/US2019/028590.

### BRIEF DESCRIPTION OF THE DRAWINGS

FIG. 1 shows an approach for personalizing recommendations for altering the micro biome of the subject.

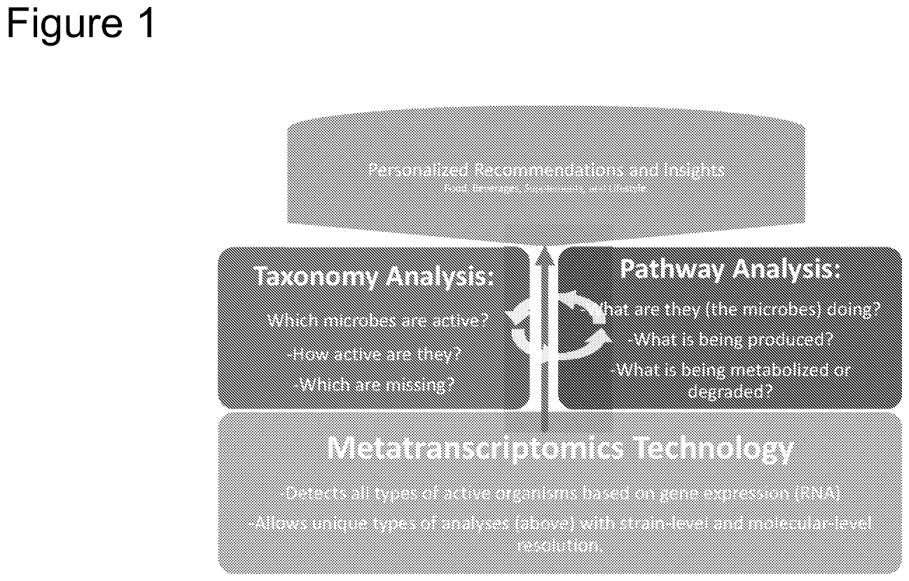

FIG. 2 shows an exemplary hierarchy of information leading to an activity score for functional category. In this example, gene activity (referred to as “molecular function”) and microbial taxa activity (referred to as “microbiome function”) provide metrics using the determination of an activity score for a functional category (referred to as “health function”) (in this case, inflammatory activity).

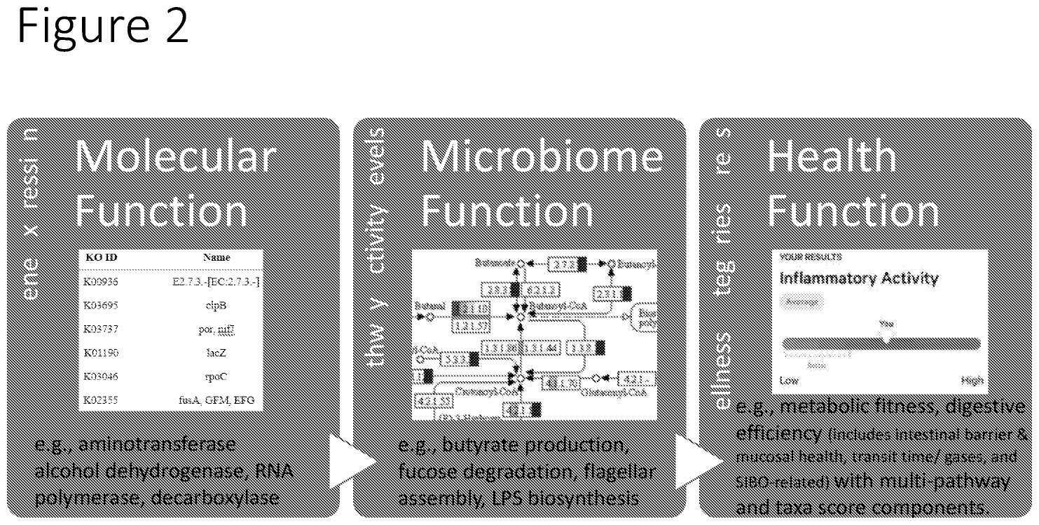

FIG. 3 shows use of a combination of pathway activity and taxa activity to inform a score for each of two functional categories, in this case pro-inflammatory and anti-inflammatory. Scores for these functional categories (“sub-scores”) are further used to determine a score for a higher order functional category. In this example, the higher order functional category is the health metric, inflammatory activity.

FIG. 4 shows development of a functional activity score for a high-level health metric, in this example, digestive efficiency. The functional activity is a composite of functional activity scores for a number of lower order functional categories. In this example, functional categories include protein fermentation, motility/gases, intestinal barrier health and SIBO-like/hypo-HCL pattern. Intestinal barrier health, in turn, is a composite of still further lower order functional categories including pro-inflammatory and anti-inflammatory components. In this example, scores for each of the intermediate functional categories in the hierarchy are based on both biochemical pathway activity (pathway component) and active taxa (taxa component).

FIG. 5 shows a protocol for analyzing a sample from a subject, determinizing a functional category having a functional activity score outside a reference range, and taking action.

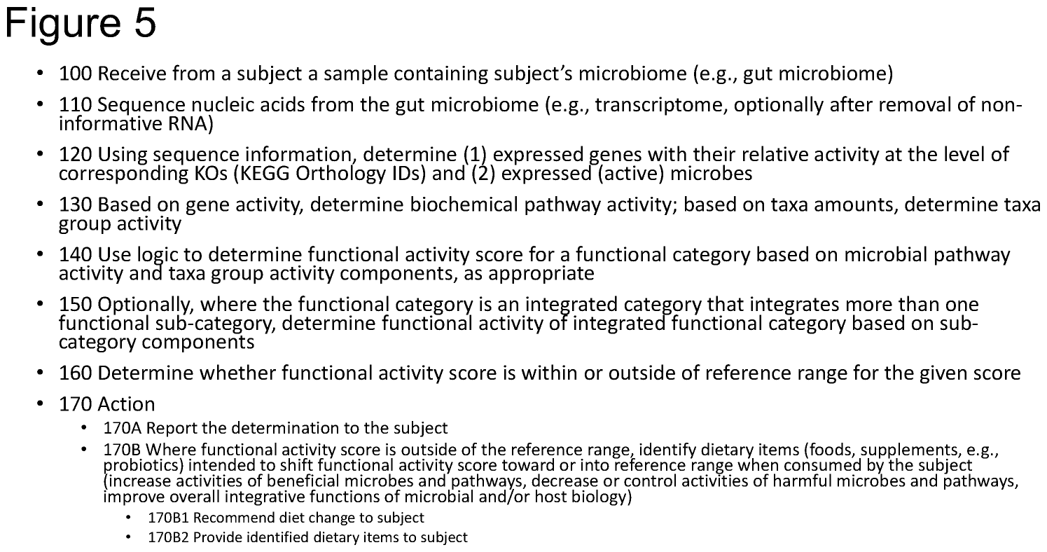

### DETAILED DESCRIPTION

Provided herein are methods to identify dysbiosis in a microbiome sample. A dysbiosis is identified through generation and combination of health scores calculated from subject microbiome test results. Imbalance in particular microorganisms or their products are associated with nutrients that will affect the growth or activity of the microorganisms. Further described are methods to generate recommendations to reestablish balance or maintain a balance in the microbiome. Dietary and supplement recommendations are generated based on the nutritional needs of the microorganisms targeted for affect.

Further provided herein are methods to identify microbial imbalances in a range of environments. Nonlimiting examples of environments provided herein comprise soil; water, water treatment facilities; environmental remediation; vaginal microbiome; prenatal care; aquatic ecosystems including algal blooms, algal crashes, and coral reefs; skin microbiome; oral microbiome; constructed environments; fermentation manufacturing including beer, wine and cheese production; etc.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The section headings used herein are for organizational purposes only and are not to be construed as limiting the subject matter described.

### II. Sample Analysis

### A. Sample Isolation

The databases, systems, and methods described herein can be used to analyze the compositions of samples. In some aspects, the databases can be used to analyze the composition of a heterogeneous microbial sample. Various types of samples are contemplated herein. These include samples from a subject, water, soil, air, surfaces, foods, etc.

The samples analyzed by the methods and systems described herein comprise heterogeneous microbial populations. Microbial communities are often made up of mixed populations of organisms, including unknown species in unknown abundances.

In some aspects, the sample can be from a species of a mammal, a species of a rodent, a species of a mouse, a species of a rat, a species of a dog, a species of a cat, a species of a hamster, a species of a monkey, a species of a pig, a species of a squirrel, a species a guinea pig, a species of a gerbil, a species of a bird, a species of a hydra, a species of a rabbit, a species of a fish, a species of a frog, a species of a cow, a species of a lamb, a species of a chicken, a species of Drosophila, a species of Xenopus, a species of horse, and a human.

A sample used in a methods described herein may be, for example, earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue (e.g., a skin biopsy), subcutaneous tissue, muscle tissue, adipose tissue, and a combination thereof.

Furthermore, a sample may be obtained from, for example, the gut, the vagina, the penis, a testicle, the cervix, the respiratory system, the ear, the skin, the rectum, the kidney, the liver, the spleen, the lung, the pancreas, the small intestine, the gallbladder, the lymph nodes, the colon, a nasal passage, the central nervous system, an oral cavity, a sinus, a nostril, the urogenital tract, an udder, an auditory canal, a breast, an open wound, the eye, fat, muscle, and combinations thereof

In some aspects, the sample can be an environmental sample or an agricultural sample. Examples include samples from soil, plant/fruit samples taken during a planting or harvesting, must, sampling of wine during alcoholic fermentation (beginning, middle and end, or depending on parameters such as alcoholic graduation, amount of sugar, density), sampling during malolactic fermentation (beginning, middle and end, or depending on amount of malic and acetic acid), barrel (beginning, middle and end, or months) and bottling. Other examples include samples from sites containing contaminants, including those associated with petroleum extraction and refining, chemical manufacturing, pesticide use, etc.

In some aspects, the sample can be one used to detect pathogenic microorganisms or microorganisms used in a bioterrorism attack. Such a sample includes analysis of microorganisms collected in an air filter or by sampling a surface. In some embodiments, the sample is a forensics sample.

Polynucleotides can be extracted directly from the sample, or cells in the sample can first be lysed to release their polynucleotides. In one method, lysing cells comprises bead beating (e.g., with zirconium beads). In another method, ultrasonic lysis is used. Such a step may not be necessary for isolating cell-free nucleic acids.

Nucleic acids can be isolated from the sample by any means known in the art. Polynucleotides can be isolated from a sample by contacting the sample with a solid support comprising moieties that bind nucleic acids, e.g., a silica surface. For example, the solid support can be a column comprising silica or can comprise paramagnetic silica beads. After capturing nucleic acids in a sample, the beads can be immobilized with a magnet and impurities removed. In another method, nucleic acids can be isolated using cellulose or polyethylene glycol.

If the target polynucleotide is RNA, the sample can be exposed to an agent that degrades DNA, for example, a DNase. Commercially available DNase preparations include, for example, DNase I (Sigma-Aldrich), Turbo DNA-free (ThermoFisher) or RNase-Free DNase (Qiagen). Also, a Qiagen RNeasy kit can be used to purify RNA.

Alternatively or in addition, a sample comprising DNA and RNA can be exposed to a low pH, for example, pH below pH 5, below pH 4 or below pH 3. At such pH, DNA is more subject to degradation than RNA.

If the target polynucleotide is RNA, the sample can be reverse transcribed into DNA. Reverse transcription generally takes place after a sample has been depleted of DNA.

In some aspects, a sample can be depleted of nucleic acids and nucleic acid species that are abundant relative to other nucleic acids in the sample. Some of the abundant nucleic acids may not be target nucleic acids (e.g., they may not encode sequence signatures or may not be informative of desired taxonomic information). The presence of these abundant nucleic acids can reduce the sensitivity of some of the methods described herein. This can be true, for example, if target or informative nucleic acids are rare relative to the abundant nucleic acids. Therefore, it can be advantageous to enrich a sample for target sequences by removing non-informative abundant sequences. Examples of sequences that can be removed include microbial ribosomal RNA, including 16S rRNA, 5S rRNA, and 23S rRNA. Other examples of sequences that can be removed include host RNA. Examples include host rRNA, such as 18S rRNA, 5S rRNA, and 28S rRNA.

As used herein, the term “non-informative RNA” refers to a form of non-target or non-analyte species of RNA. Non-informative RNA species can include one or more of: human ribosomal RNA (rRNA), human transfer RNA (tRNA), microbial rRNA, and microbial tRNA. Non-informative RNA species can further comprise one or more of the most abundant mRNA species in a sample, for example, hemoglobin and myoglobin in a blood sample.

Methods of enriching nucleic acid samples include the use of oligonucleotide probes. Such probes can be used for either positive selection or negative selection. Such methods often reduce the amount of non-target nucleotides.

If the target polynucleotide is DNA, then DNA can be isolated with silica, cellulose, or other types of surfaces, e.g., Ampure SPRI beads. Kits for such procedures are commercially available from, e.g., Promega (Madison, Wisc.) or Qiagen (Venlo, Netherlands).

The isolated nucleic acids are generally sequenced for subsequent analysis. The methods described herein generally employ high throughput sequencing methods. As used herein, the term “high throughput sequencing” refers to the simultaneous or near simultaneous sequencing of thousands of nucleic acid molecules. High throughput sequencing is sometimes referred to as “next generation sequencing” or “massively parallel sequencing.” Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing (PacBio), and nanopore DNA sequencing (e.g., Oxford Nanopore).

### B. Nucleic Acid Sequencing and Transcriptome Sequence Preprocessing

Also provided herein are methods of analyzing nucleic acids, such as genomic DNA and RNA transcripts, in a heterogeneous microbial sample. The RNA transcripts can be part of a transcriptome for a cell or cells in the heterogeneous microbial sample. The methods generally include isolating and sequencing the RNA found in a sample as described above.

The sequences obtained from these methods can be preprocessed prior to analysis. If the methods include sequencing a transcriptome, the transcriptome can be preprocessed prior to analysis. In one method, sequence reads for which there is paired end sequence data are selected. Alternatively or in addition, sequence reads that align to a reference genome of the host are removed from the collection. This produces a set of host-free transcriptome sequences. Alternatively or in addition, sequence reads that encode non-target nucleotides can be removed prior to analysis. As described above, non-target nucleotides include those that are over-represented in a sample or non-informative of taxonomic information. Removing sequence reads that encode such non-target nucleotides can improve performance of the systems, methods, and databases described herein by limiting the sequence signature database to open reading frames can the size of the database, the amount of memory required to run the sequence signature generation analysis, the number of CPU cycles required to run the sequence signature generation analysis, the amount of storage required to store the database, the amount of time needed to compare sample sequences to the database, the number of alignments that must be performed to identify sequence signatures in a sample, the amount of memory required to run the sequence signature sample analysis, the number of CPU cycles required to run the sequence signature sample analysis, etc.

In certain embodiments, quantitative measures of gene activity and microbial taxa are determined using the transcriptome of a microbiome of the subject. The transcript on includes RNA that is transcribed from cells in a sample, in particular, microbial cells. In certain embodiments transcriptome analyzed comprises or consists essentially of messenger RNA, in particular, microbial mRNA. In certain embodiments non-informative RNA is removed from the transcriptome before analysis. In particular, ribosomal RNA can be removed from the transcriptome. Accordingly, taxonomic analysis can be performed on mRNA rather than rRNA.

### C. Taxonomic Identification

### 1. Paired End Alignment

To determine the identity of one or more organisms present in a sample at a specific taxonomic level, paired-end (or optionally single) transcriptome reads from that sample are aligned to the library of taxonomic signatures as described herein at that specific taxonomic level. Sequences can be aligned using, for example, the BWA aligner with the mem algorithm. (Li H. (2013) “Aligning sequence reads, clone sequences and assembly contigs with BWA-,” arXiv:1303.3997v1 [q-bio.GN].) BWA is often run with a minimal seed alignment length of 30 nt, but other BWA parameters such as the mis-match penalty can be modulated, as can downstream filters. Global thresholds for sensitivity and specificity can be tuned at this level by modulating the BWA parameters during model training after taxonomic signature generation on test datasets of known composition. These values can then be applied during the identification step. The best unique alignment of a read or read pair to a unique genome signature at a specific taxonomic level identifies that taxonomic member as being present. Some organisms will be identified to the strain taxonomic level while others may only be identified to the genus level (or higher) depending on the nature of distinct sequences available in the database to make an accurate determination. In some aspects, a microorganism or a taxon is identified as being present in the sample if 1, 2, 3, 4, 5, 6, 7, 8, 9, 10, 15, 20, 25, 30, 35, 40, 45, 50, 60, 70, 80, 90, 100 or more sequence signatures corresponding to the taxon are detected in the sample. In some aspects, a microorganism or a taxon is identified as being present in the sample if 1, 2, 3, 4, 5, 6, 7, 8, 9, 10, 15, 20, 25, 30, 35, 40, 45, 50, 60, 70, 80, 90, 100 or more sequence reads are detected for a sequence signature in the sample. In some aspects, a read is determined to match a sequence signature if the read is 90%, 95%, 96%, 97%, 98%, 99%, 99.5%, 99.9% or 100% identical to at least a portion of the sequence signature or the entire sequence signature. In some aspects, a read is determined to match a sequence signature if the sequence signature is 90%, 95%, 96%, 97%, 98%, 99%, 99.5%, 99.9% or 100% identical to at least a portion of the read or the entire read. In a preferred embodiment, calling or identifying a microorganism as being present at one or more taxonomic levels requires at last 25 reads of at least 100 nucleotide and a mismatch rate no greater than 2% when aligned against the curated database/library of unique genome signatures.

### 2. Use of Flanking Sequences

In another embodiment, transcriptome sequences that align to one portion of the unique genome signature database/library but do not align to sequences flanking the first portion in that alignment are eliminated.

In one version of this embodiment, identification of organisms on a particular taxonomic member at a taxonomic level can be determined by making use of sequences that flank sequences in the taxonomic signature. That is, the method can make use of sequences of the genome removed from the genome in the generation of the taxonomic signature. In this method transcriptome sequences are often first aligned to the taxonomic signature sequences. Then, portions of the aligned transcriptome sequences that do not, themselves, align with the signature sequences can be compared to the sequences that flank the signature sequences. If there is insufficient homology between these flanking sequences, the entire sequence read can be removed from the alignment protocol. The minimum level of homology required for a sequence to remain in alignment can be, for example, at least 90%, at least 95%, at least 98%, at least 99% or 100%.

### D. Microorganism Quantitation

The methods, systems, and databases described herein can also be used to quantify a characteristic in a heterogeneous sample. In one aspect, provided herein are methods for quantifying microorganisms in a sample. Alternatively or in addition, provided herein are methods for quantifying a particular biochemical activity associated with a heterogeneous microbial sample or a microorganism contained therein. Transcriptome sequences from the sample can be mapped to the ORF library described above. In some aspects, ORF library can be used to infer taxonomic information about the organisms present in the sample. Alternatively or in addition, sequences that map to the library can be annotated to indicate information such as gene identity and gene function.

Quantification of abundance can be done by computer by summing up the non-overlapping length of profiles found for a taxonomic member (Linear Length or L), then determining the read coverage across that length (Linear Depth of Coverage or DOC). In some aspects, the non-overlapping length is or comprises a sequence signature informative of taxonomic information.

Normalizing over the sum of all DOCs for a specific taxonomic level allows one to arrive at the relative abundance (RA) of that taxonomic member. In one embodiment, normalization comprises: determining the number of base pairs contained in a particular ORF, determining an average depth of coverage for the entire length of the ORF using the number nucleotides contained in sequence reads corresponding to the ORF and the length of the ORF, and determining the proportion of all sequence reads that correspond to the ORF.

Normalization can comprise determining an average depth of coverage for ORFs identified in the sequencing data, summing the averages to generate a total depth of coverage, and dividing the depth of coverage for an ORF of interest by the total depth of coverage. Such a method can be used to determine a relative amount or proportion of sequence reads for the target ORF in the sequencing data. Such methods can also account for the relative differences in lengths of ORFs, allowing for more direct comparisons, because the methods can use the depth of coverage for an ORF rather than the total number of bases read. Thus, a number of base reads in a 1,000 bp ORF would have half the total depth of coverage compared to the same number of base reads in a 500 bp ORF. The relative amount or proportion of the ORF in the sample can be used to infer the relative activity of the target ORF.

In some aspects, the amount or relative amount can be compared to a reference value. Examples of a reference value include a normal value and a cutoff value that is a specified distance from a normal value (e.g., in units of standard deviation). The reference value may be determined from a different sample from the same subject (e.g., when the subject was known to be healthy or prior to administration of the food, supplement, or medication). The reference value may also be determined from one or more samples from other healthy subjects. The reference value may be an absolute or relative value.

The measure of gene expression at each taxonomic level can be calculated based on the Reads Per Kilobase (RPK) of transcript per Million mapped reads. Additional filters can be applied at this step on read count, L, DOC, or RA with thresholds determined during the model training step. Note this relative abundance will be for the DNA or RNA fraction of that taxonomic member in the sample at time of library prep. If the source was RNA, then the relative abundance calculated can correspond to the relative activity of that organism in the sample (e.g., gene expression levels). Alternatively or in addition, if the source was RNA, then the relative abundance calculated for an ORF can correspond to the relative activity of that ORF, including the activity or pathway. If the source was DNA, one relates to relative abundance of that organism assuming a single genome copy per organism. The taxonomic relative activity can be quantified by finding the median or mode of non-zero Reads Per Kilobase (RPK) of transcript per Million mapped reads (RPKM values) and inversing scaling by the fraction of active genes.

The output of this process can be a report that indicates for a subject sample the taxa of microorganisms in the sample. If the taxonomic identity of the sample cannot be identified at a particular taxonomic level the report can indicate the intensity at the next highest taxonomic level. The report can also indicate quantitative information about the sample this can include, for example, the relative amounts of different microorganisms in the sample. It can also indicate relative activity of microorganisms in the sample based on relative gene expression. This can include, for example, types of genes that are either expressed in high amounts or alternatively, in low amounts. Alternatively or in addition, the report can indicate the identity and relative amounts of biochemical activities in the sample. The report can indicate changes as to biochemical activity in the sample over time, such as during a time course. The report can indicate differences between samples, including samples collected from the same source at different times. The source can be a subject, including a human subject.

In some aspects, limiting the sequence signature database to open reading frames can reduce the size of the database, the amount of memory required to run the sequence signature generation analysis, the number of CPU cycles required to run the sequence signature generation analysis, the amount of storage required to store the database, the amount of time needed to compare sample sequences to the database, the number of alignments that must be performed to identify sequence signatures in a sample, the amount of memory required to run the sequence signature sample analysis, the number of CPU cycles required to run the sequence signature sample analysis, etc.

Reports can sometimes be output to paper, a screen, or a database. Reports can also be stored for later analysis or viewing. Reports can be sent to third parties, such as subjects, healthcare professionals, customers, collaborators, etc.

### E. Gene Activity Quantitation

The methods, systems and databases herein can be used to identify activity of a gene or a biochemical pathway present in the sample. In some embodiments, the methods include aligning sequencing reads to a database comprising open reading frame information that is associated with a particular biochemical activity or pathway, as described above. Some of such methods can include identifying taxonomic information for a sequence. Examples include the VIOMEGA algorithm (see WO 2018/160899 (Vuyisich et al.) or GOTTCHA algorithm, which detects sequence signatures that identify nucleic acids as originating from organisms at various taxonomic levels. Nucleic Acids Res. 2015 May 26; 43(10): e69. Other methods include MetaPhlAn, Bowtie2, mOTUs, Kraken, and BLAST. Some of such methods do not include identifying taxonomic information for the sequence, but instead may identify the biochemical activity, pathway, protein, functional RNA, product, or metabolite associated with a particular sequence read or sequence signature.

“Gene activity” or “activity of a gene” is a generally a function of transcription, e.g., the quantity of RNA in a sample encoding the gene. This can be done at any taxonomic level. For example, gene activity could be a measure of activity of the gene in a single species, or it could be activity of the gene across organisms belonging to a common genus, class, order or phylum.

### III. Functional Categories and Functional Activity Scores

Functional categories include biological or health functions or conditions at the cellular, organ or organismal level. Functional categories typically include, as components, one or more pathways and/or one or more taxa groups. Functional categories are assigned functional activity scores based on data derived from a subject. Functional activity scores represent quantitative measures of activity.

### A. Pathways

Biological pathways are sequences of proven molecular events (such as enzymatic reactions or signal transduction or transport of substances or morphological structure changes) that lead to specific functional outcomes (such as secretion of substances, sporulation, biofilm formation, motility). Many biological pathways are known in the art, and examples can be found on the web at wikipathways.org/index.php/WikiPathways, pathwaycommons.org, and proteinlounge.com/Pathway/Pathways.aspx. Manual expert curation of scientific literature also can be used to reconstruct or create custom biological pathways. Biological pathways can include a number of genes that encode peptides or proteins, which play specific signaling, metabolic, structural or other biochemical roles in order to carry out various molecular pathways. As used herein, the terms “biochemical activity” and “biochemical pathway activity” refer to activity of a biochemical pathway.

Pathways of interest include, without limitation, secondary bile acid pathway, primary bile acid pathway, butyrate pathway, methanogenesis pathway, acetate pathway, propionate pathway, branch chain amino acid pathway, long chain fatty acid metabolism pathway and long chain carbohydrate metabolic pathway.

### B. Taxa Groups

Microbial taxa includes taxonomic designation at any taxonomic level, e.g., species, genus, order or phylum. Active microbial taxa are taxa that are not really present but that are metabolically active, e.g., as measured by transcriptional levels of the microbial genome. Groups of microbial taxa whose activity contribute to functional activity in a functional category are referred to herein as “taxa groups”. So, for example, pro-inflammatory taxa group can comprise one or more of: proteobacteria, opportunistic bacteria or pathogens, viruses; anti-inflammatory taxa group can comprise one or more of: butyrate producers, Lactobacilli and Bifidobacteria; intestinal barrier disruptors taxa comprise one or more of: Ruminococcos torques, Ruminococcus gnavus, Serratia, Sutterella, and other mucus-degrading or epithelial layer-disrupting organisms.

Taxa groups of interest include, without limitation, Prevotella (genus)/Bacteroides (genus) ratio, Eubacterium rectale (species), Eubacterium eligens (species), Faecalibacterium prausnitzii (species), Akkermansia muciniphila (species), metabolic-related probiotic species (functional group), Roseburia (genus), Bifidobacterium (genus), Lactobacillus (genus), Clostridium butyricum (species), Allobaculum (genus), Firmicutes (phylum)/Bacteroidetes (phylum) ratio, Lachnospiraceae (family), Enterobacteriaceae (family), Ralstonia pickettii (species), Bilophila wadsworthia (species).

### C. Functional Categories

A functional category can involve any function related to health or wellness. Functional categories can embrace health parameters, health indicators, health conditions and health risks. Functional categories include, without limitation, Examples of functional categories include, without limitation, inflammatory activity, metabolic fitness, digestive efficiency, intestinal barrier health, protein fermentation, gas production, microbial richness, butyrate production pathway, LPS biosynthesis pathway, methane gas production pathway, sulfide gas production pathway, flagellar assembly pathway, ammonia production pathway, putrescine production pathway, oxalate metabolism pathway, uric acid production pathway, salt stress pathway, biofilm chemotaxis in virulence pathway, TNA production pathway, primary bile acid pathway, secondary bile acid pathway, acetate pathway, propionate pathway, branch chain amino acid pathway, long chain fatty acid metabolism pathway, long chain carbohydrate metabolic pathway, detoxification potential (ability of microbiome to detoxify the body), gut neuro-balance (impact of microbiome on the brain, e.g., by production of neurotransmitters), neurological health, cardiovascular health, hormonal balance, musculoskeletal health, hepatic function, urogenital health, mitochondrial activity, immune function, gastrointestinal health, diabetes, skin conditions and infectious disease.

Functional categories also include categories that may contribute to more general categories, such as wellness, stress, anxiety, allergies, autoimmune condition, leaky gut, insulin resistance, metabolic syndrome, metabolic type, insomnia and, skin conditions.

Functional categories typically, but not always, integrate both pathway activity and taxa activity components.

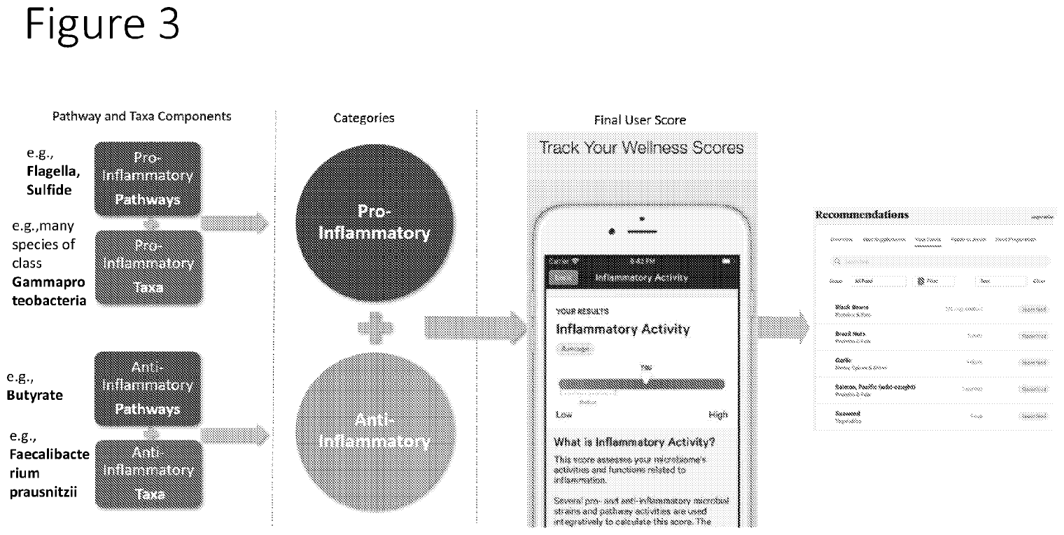

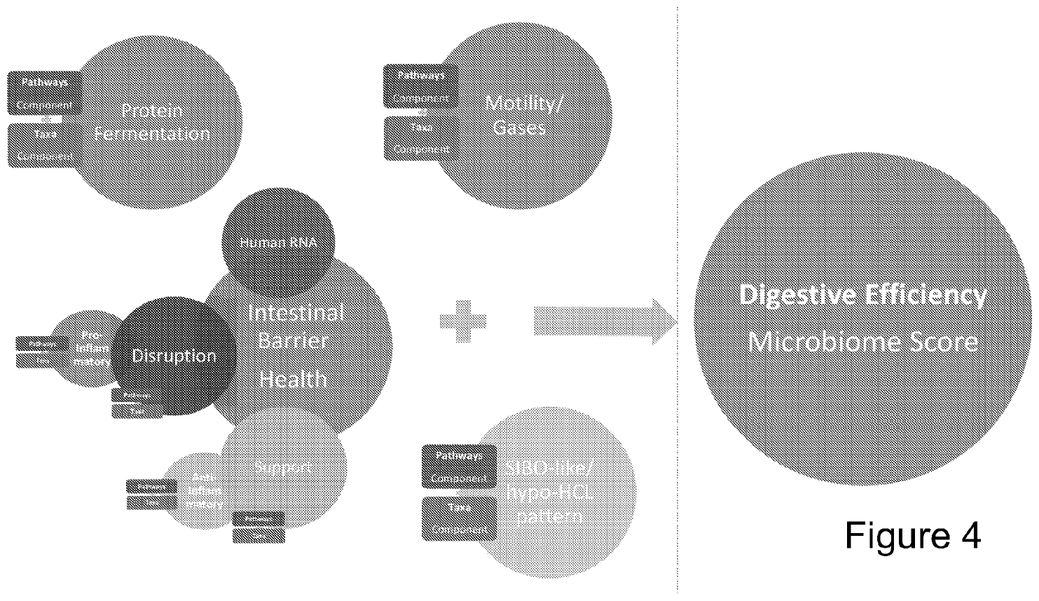

Functional categories can be hierarchical in nature, with functional categories at lower levels in the hierarchy being aggregated into functional categories at higher levels in the hierarchy. For example, at a lowest level a single biochemical pathway or a group of microbial taxa can serve as a function category. Combinations of pathways and microbial taxa groups can be integrated into higher level categories. This includes, for example, a plurality of pathways, a plurality of taxa groups or at least one pathway and at least one taxa group. Referring to FIG. 3, inflammatory activity is a functional category that aggregated pro-inflammatory and anti-inflammatory components. Each of these components represents a functional category. In turn, each of the pro-inflammatory and anti-inflammatory categories aggregated scores from biochemical pathways and taxa groups. Referring to FIG. 4, a number of functional categories can be aggregated into a higher order functional category, in this case, digestive efficiency. More specifically, in this example, digestive efficiency aggregated scores from the categories protein fermentation, motility/gases, intestinal barrier health and SIBO-like/hypochlorhydrea pattern. While the final aggregated functional category is provided with a functional activity score, each sub functional category which is comprised within the highest functional category may itself be provided with a discrete score or other logic may be used to aggregate functional activities of the subcategories into the topmost functional category.

### D. Functional Activity Score

A “functional activity score” refers to a quantitative measure assigned to an activity or state of a functional category. A functional activity score can be assigned to a functional category in a subject based on data from the microbiome, such as meta-transcriptomic data. A functional activity score can be given as within or outside a reference value, such as a range. The reference value can be derived from values across a population of subjects. For example, the reference range may constitute a statistical range within the population, such as a standard deviation from the mean. Alternatively, the reference range may be determined by expert analysis, by logic and/or with reference to literature sources. The value can be given as “low” “medium” or “high”, with “medium” constituting the reference range. Both “low” and “high” may be outside the reference range.

Quantitative measures can be given as a discrete or continuous range. Quantitative measures can be absolute numbers or relative amounts, such as normalized amounts. Quantitative measures include statistical measures such as mean, variance and standard deviation. For example, a quantitative measure can be a number, a degree, a level or bucket. A number can be a number on a scale, for example 1-10. Alternatively, the quantitative measure can embrace a range. For example, ranges can be high, medium and low; severe, moderate and mild; or actionable and non-actionable. Buckets can comprise discrete numerals, such as 1-3, 4-6 and 7-10. quantitative measure (number, range, relative amount, etc.).

### IV. Health Recommendations

The methods described herein may be used to identify a dysbiosis in a microbiome sample. A dysbiosis can be an overabundance from normal levels of a microorganism typically present in a healthy microbiome. A dysbiosis can be a depletion from normal levels of a microorganism typically present in a healthy microbiome. A dysbiosis can be a colonization of a microorganism typically not present in a healthy microbiome. A dysbiosis can be an abnormal increase, decrease, presence, or absence of a biochemical activity in a microbiome.

Dysbiosis in the microbiome is characterized through health scores. In some aspects, a score is calculated relative to a healthy population. In some aspects, a score is calculated relative to a sampling of the microbiome at a different time point. In some aspects, more than one score is combined to generate a health score. In some aspects, the score identifies a dysbiosis in the subject microbiome. In some aspects described herein, one or more symptoms are identified in a subject. In some aspects, a correlation is calculated between the dysbiosis in the microbiome and the symptom identified in the patient.

A dysbiosis in a microbiome is further defined by imbalance in particular microorganisms. In methods described herein, microorganisms are identified that affect the dysbiosis in the microbiome or symptom identified in the subject. In some aspects, the dysbiosis or symptom is ameliorated by an increase in identified microorganisms. In some aspects, the dysbiosis or symptom is ameliorated by a decrease in identified microorganisms. In some aspects, the dysbiosis or symptom is ameliorated by an increase in a product or a biochemical activity of the identified microorganism. In some aspects, the dysbiosis or symptom is ameliorated by a decrease in a product or a biochemical activity of the identified microorganism. In some aspects, as described herein, the product of the identified microorganism is a metabolite.

A dysbiosis can be corrected by affecting the growth or activity of the microorganisms. In methods described herein, substances are disclosed that influence the growth, activity, or expression of product of an identified microorganism. In some aspects, the substance is a nutrient, micronutrient, supplement, food, or probiotic. A nutrient includes a substance used by an organism to survive, grow, and reproduce. In some aspects, nutrients may include macronutrients or micronutrients. Macronutrients include, for example, carbohydrates, fats, proteins, water, calcium, sodium, potassium, magnesium, chloride ions, phosphorus, and sulfur. Micronutrients include, for example, a mineral, a trace mineral, a vitamin, a biochemical substrate, or any combination thereof. A mineral may be calcium, magnesium, sulfur, or any combination thereof. A trace mineral may be iron, chromium, copper, fluoride, iodine, manganese, molybdenum, selenium, zinc, or any combination thereof. A vitamin may be thiamin (B1), riboflavin (B2), niacin, Vitamin B6, cobalamin (B12), folate, ascorbic acid, Vitamin A, Vitamin D, Vitamin E, Vitamin K, or any combination thereof.

Substances to affect the growth or activity of microorganisms can be found in a variety of foods and supplements. Supplements can include vitamins, minerals, probiotics, and prebiotics. Described herein are methods comprising a recommendation to improve a health characteristic in a subject. In some aspects, the recommendation comprises administration of one or more microorganisms or substances identified to ameliorate the dysbiosis or symptom identified in the subject. In some aspects, the recommendation comprises a food recommendation, wherein the food comprises a micronutrient or substance known to affect the growth or activity of a microorganism. For example, butyrate in the gut protects lining of the intestine, reduces inflammation and prevents “leaky gut.” Low levels of butyrate can be addressed by supplementing the system with prebiotics, or nutrients to aid growth of butyrate-producing microbes. Alternatively, butyrate-producing microbes can be administered to the system as a probiotic treatment. Third, the system can be administered a butyrate supplement. Finally, a combination of any of the foregoing alternatives and be administered in concert or consecutively.

In aspects described herein, a recommendation is consistent with the presence of a microorganism or biochemical activity. In some aspects, an identified microorganism or biochemical activity is desirable or beneficial to the subject, in which case a recommendation is made to benefit or increase the quantity of microorganism or biochemical activity. In some aspects, an identified microorganism or biochemical activity is undesirable or detrimental to the subject, in which case a recommendation is made to suppress or decrease the quantity of microorganism or biochemical activity.

Activity of microorganisms can be measured by expression of target genes. In aspects described herein, biochemical activity in a microbiome sample is described by mapping sequencing data to an ORF library, for example, the KEGG Orthology (KO) database. Sequencing data can then be correlated with families of genes and specific organisms can be identified.

In aspects described herein, a recommendation is optimized to correct dysbioses or symptoms with conflicting requirements for amelioration. In some aspects, a recommendation provides aspects to increase growth or activity of some microorganisms and reduce growth or activity of other microorganisms. In some aspects, a recommendation is based on a prioritization of biological issues.

Food or dietary recommendations are generated through use of a curated food ontology. In methods described herein, foods or supplements which contain nutrients or substances known to improve the balance of a microbiome are selected from a food ontology database. In some aspects, a food is selected based on the nutrient or substance provided therein, which can improve a balance in a microbiome.

Thus, in some aspects, the methods include sequencing a sample from a subject in order to assess the composition or activity of a microbiome of the subject, detecting a dysbiosis in the subject's microbiome using the sequencing data, and providing a recommendation, a food, or a supplement to the subject to correct the dysbiosis or improve a health characteristic in the subject.

In embodiments described herein, health characteristics addressed by this method comprise imbalances of the immune system, the endocrine system, the digestive system, the metabolic system, the neurological system, psychiatric disorders, or any combination thereof

In some aspects described herein, characteristics of the immune system may include an immune tolerance, an autoimmune disease or condition, an inflammation, an infection, or any combination thereof. In some aspects, immune tolerance may include peripheral tolerance, central tolerance, or tumor immunotolerance. In some aspects, an autoimmune disease or condition may include celiac disease, diabetes mellitus type 1, sarcoidosis, systemic lupus erythematosus (SLE), Sjögren's syndrome, eosinophilic granulomatosis with polyangiitis, Hashimoto's thyroiditis, Graves' disease, idiopathic thrombocytopenic purpura, Addison's disease, rheumatoid arthritis (RA), ankylosing spondylitis, polymyositis (PM), dermatomyositis (DM) and multiple sclerosis (MS). In some aspects, infection may include an infection by a pathogen, for example a bacteria, a virus, a fungi, or a prion. In further aspects, characteristics of the immune system may include an inflammation.

In some aspects described herein, characteristics of the endocrine system may include function of the pineal gland, pituitary gland, thyroid gland, parathyroid gland, thymus gland, adrenal gland, pancreas, ovaries, testes, or any combination thereof. In some aspects, characteristics of the endocrine system may include a hormone level. In some aspects, the hormone may be a eicosanoid, a steroid or an amino acid/protein derivative.

In some aspects described herein, characteristics of the digestive system may include inflammatory bowel disease (IBD), ulcerative colitis, Crohn's disease, irritable bowel syndrome (IBS), necrotizing enterocolitis, colic, constipation, lactose intolerance, or any combination thereof

In some aspects described herein, characteristics of the metabolic system may include digestion, oxidative phosphorylation, chemolithotrophy, anabolism, and xenobiotic metabolism.

In some aspects described herein, characteristics of the neurological system may include ALS, arteriovenous malformation, brain aneurism, brain tumors, epilepsy, headache, memory disorders, multiple sclerosis, Parkinson's Disease, peripheral neuropathy, and stroke.

In some aspects described herein, characteristics of psychiatric disorders may include anxiety or fear; mood disorder, including depression and bipolar disorder; psychotic disorders such as schizophrenia and delusional disorder; personality disorders including paranoid, borderline, and obsessive-compulsive disorders; eating disorders; sleep disorders; and sexual disorders.

Recommendations for altering diet can be provided to a subject in electronic or paper format. Electronic communications can be communicated to the subject over a communications network to an electronic device accessible by the subject. Data can be transmitted electronically, e.g., over the Internet. Communication may be, for example, in the form of information provided on a password-protected website accessible by the subject. Alternatively, communication may be by email or text message. Electronic devices accessible by the subject can include, for example, computers connected to the Internet, smart phones (e.g., iPhone® or Samsung Galaxy®), or a wearable device (e.g., Fitbit® or Garmin®). Electronic communication can be, for example, over any communications network include, for example, a high-speed transmission network including, without limitation, Digital Subscriber Line (DSL), Cable Modem, Fiber, Wireless, Satellite and, Broadband over Powerlines (BPL). Information can be transmitted to a modem for transmission e.g. wireless or wired transmission, to a computer such as a desktop computer. Alternatively, reports can be transmitted to a mobile device. Reports may be accessible through a subscription program in which a user accesses a website which displays the report. Reports can be transmitted to an electronic device accessible by the user. This could be, for example, a personal computer, a laptop, a smart phone or a wearable device, e.g. worn on the wrist.

The diet of a subject refers to the total kind and quantities of food, supplements, probiotics, and medicines consumed by a subject over a defined period of time e.g., over the course of about a day, about a week, about a month or about a year. A diet can be further defined in terms of macronutrient and micronutrient content. Macronutrients include, for example, carbohydrates, fiber (generally indigestible carbohydrates), proteins, and fats. Macronutrients used in this analysis can include any suitable group of macronutrients, such as carbohydrates, fiber (generally indigestible carbohydrates), proteins, and fats. Micronutrients include, for example, a mineral, a trace mineral, a vitamin, a biochemical substrate, or any combination thereof. A mineral may be calcium, magnesium, sulfur, or any combination thereof. A trace mineral may be iron, chromium, copper, fluoride, iodine, manganese, molybdenum, selenium, zinc, or any combination thereof. A vitamin may be thiamin (B1), riboflavin (B2), niacin, Vitamin B6, cobalamin (B12), folate, ascorbic acid, Vitamin A, Vitamin D, Vitamin E, Vitamin K, or any combination thereof.

Altering the diet of the subject can alter activity in any number of functional categories, resulting in desired changes in either specific microbial pathways and/or broader biological functions of the microbiome or of the host (based on the microbiome). In general, an aim of altering diet is to rebalance the microbiome of a subject such that functional activity scores shift toward or into the reference range. This can be accomplished by several means. One method is to provide dietary items that promote the production of useful nutrients by the gut microbes. Another method is to reduce or avoid foods high in nutrients that are used by the microbes to produce harmful products. Another method is to provide a food or supplement containing probiotic microbes that produce beneficial nutrients or “overpower” the harmful microbes or the activity levels of microbial pathways that yield harmful products. Another method is to provide the beneficial macro- and/or micronutrients directly in the available forms of diet and supplement recommendations.

### V. Food and Supplement Delivery

In another aspect, after determining that a functional activity score of a subject is outside reference range, one or more of a food, a supplement, a probiotic or a medicine can be identified which, when included in the diet of the subject, shifts the functional activity score toward or into the reference range. So, for example, if it is determined that a subject has an inflammatory activity score that is high compared with the reference range, dietary items that will decrease the inflammatory activity score can be identified. These might include, for example, foods or supplements high in antioxidants or probiotics including microbes that depress pro-inflammatory biochemical pathways, such as the butyrate pathway.

These dietary items can be delivered to a subject, for example, via common carrier. Such items can be provided in a kit, which typically includes a collection of items intended for use together. Kits can include containers to hold dietary items. Containers, themselves, can be placed into a shipping container, such as a box or a bag. The container can be transmitted by hand delivery or by a common carrier, such as a national postal system or a delivery service such as UPS or FedEx. Kits can also typically include written recommendations or instructions for use.

### VI. Exemplary Embodiments

1. A method of improving a health characteristic in a subject comprising: obtaining nucleic acids from a sample from the subject; obtaining sequence information representative of the nucleic acids; assigning a taxonomic designation to a portion of the nucleic acids having sequence signatures informative of the taxonomic designation; quantifying an amount of microorganisms in the sample corresponding to the taxonomic designation; comparing the quantity of microorganisms in the sample corresponding to the taxonomic designation to a reference value for the microorganism corresponding to the taxonomic designation; identifying a nutrient that alters the growth of microorganisms corresponding to the taxonomic designation; and recommending or administering to the subject a food comprising the nutrient that alters the growth of microorganisms corresponding to the taxonomic designation. 2. The method of embodiment 1, wherein quantifying an amount of microorganisms comprises quantifying relative amounts of a plurality of microorganisms. 3. The method of embodiment 2, wherein administration of the food adjusts the relative amounts of microorganisms in the subject to approach the reference value. 4. The method of embodiment 1, wherein the reference value represents a benchmark for the health characteristic. 5. The method of embodiment 1, wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. 6. The method of embodiment 5, wherein the sample comprises feces. 7. The method of embodiment 1, wherein the health characteristic comprises an immune system function, an endocrine system function, a digestive system function, a metabolic function, a mental health characteristic, or any combination thereof. 8. The method of embodiment 7, wherein the immune system function comprises an immune tolerance, an autoimmune disease, an inflammation, an infection, or any combination thereof. 9. The method of embodiment 8, wherein the autoimmune disease comprises celiac disease, diabetes mellitus type 1, Graves' disease, inflammatory bowel disease, multiple sclerosis, psoriasis, rheumatoid arthritis, systemic lupus erythematosus, or any combination thereof. 10. The method of embodiment 8, wherein the infection comprises a respiratory infection, a urinary tract infection, a Clostridium difficile infection, or any combination thereof. 11. The method of embodiment 7, wherein the endocrine system function comprises a thyroid function, a hormone level, a cholesterol level change, or any combination thereof. 12. The method of embodiment 11, wherein the hormone level is a cortisol level, an estrogen level, an insulin level, a leptin level, a melatonin level, an oxytocin level, a testosterone level, or any combination thereof. 13. The method of embodiment 7, wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. 14. The method of embodiment 7, wherein the metabolic function comprises glucose metabolism, weight gain, weight loss, insulin level, fat mass accumulation, or any combination thereof. 15. The method of embodiment 7, wherein the mental health characteristic comprises anxiety, depression, Parkinson's disease, autism spectrum disorder, or a combination thereof. 16. The method of embodiment 1, wherein the nucleic acids obtained comprise RNA, and wherein the RNA comprises mRNA, tRNA, rRNA, snRNA, or any combination thereof. 17. The method of embodiment 1, wherein the nucleic acids obtained comprise DNA. 18. The method of embodiment 1, wherein the food comprises a micronutrient. 19. The method of embodiment 18, wherein the micronutrient increases growth of a microbial taxon. 20. The method of embodiment 18, wherein the micronutrient increases a quantity of a microbial taxon. 21. The method of embodiment 18, wherein the micronutrient suppresses growth of a microbial taxon. 22. The method of embodiment 18, wherein the micronutrient decreases a quantity of a microbial taxon. 23. The method of embodiment 18, wherein the micronutrient increases a quantity of a first microbial taxon that competes with a second microbial taxon. 24. The method of embodiment 18, wherein the micronutrient increases a quantity of a first microbial taxon that increases a quantity of a second microbial taxon. 25. The method of embodiment 18, wherein the micronutrient increases the bioavailability of a substance that improves health. 26. The method of embodiment 18, wherein the micronutrient increases the uptake of a substance that improves health. 27. The method of embodiment 18, wherein the micronutrient comprises a mineral, a trace mineral, a vitamin, a biochemical substrate, or any combination thereof. 28. The method of embodiment 27, wherein the mineral is calcium, magnesium, sulfur, or any combination thereof. 29. The method of embodiment 27, wherein the trace mineral is iron, chromium, copper, fluoride, iodine, manganese, molybdenum, selenium, zinc, or any combination thereof. 30. The method of embodiment 27, wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof.

31. A method of improving a health characteristic in a subject comprising: obtaining nucleic acids from a sample from the subject; obtaining sequence information representative of the nucleic acids; assigning a taxonomic designation to a portion of the nucleic acids having sequence signatures informative of the taxonomic designation; quantifying an amount of microorganisms in the sample corresponding to the taxonomic designation; comparing the quantity of microorganisms in the sample corresponding to the taxonomic designation to a reference value for the microorganism corresponding to the taxonomic designation; identifying a nutrient that alters the growth of microorganisms corresponding to the taxonomic designation; and recommending or administering to the subject a supplement comprising the nutrient that alters the growth of microorganisms corresponding to the taxonomic designation. 32. The method of embodiment 31, wherein quantifying an amount of microorganisms comprises quantifying relative amounts of a plurality of microorganisms. 33. The method of embodiment 32, wherein administration of the supplement adjusts the relative amounts of microorganisms in the subject to approach the reference value. 34. The method of embodiment 31, wherein the reference value represents a benchmark for the health characteristic. 35. The method of embodiment 31, wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. 36. The method of embodiment 35, wherein the sample comprises feces. 37. The method of embodiment 31, wherein the health characteristic comprises an immune system function, an endocrine system function, a digestive system function, a metabolic function, a mental health characteristic, or any combination thereof. 38. The method of embodiment 37, wherein the immune system function comprises an immuno-tolerance, an autoimmune disease, an inflammation, an infection, or any combination thereof. 39. The method of embodiment 38, wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. 40. The method of embodiment 38, wherein the infection comprises a respiratory infection, a urinary tract infection, a Clostridium difficile infection, or any combination thereof. 41. The method of embodiment 37, wherein the endocrine system function comprises a thyroid function, a hormone level, a cholesterol level change, or any combination thereof. 42. The method of embodiment 41, wherein the hormone level is a cortisol level, an estrogen level, an insulin level, a leptin level, a melatonin level, an oxytocin level, a testosterone level, or any combination thereof. 43. The method of embodiment 37, wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. 44. The method of embodiment 37, wherein the metabolic function comprises glucose metabolism, weight gain, weight loss, insulin level, fat mass accumulation, or any combination thereof. 45. The method of embodiment 37, wherein the mental health characteristic comprises anxiety, depression, Parkinson's disease, autism spectrum disorder, or a combination thereof. 46. The method of embodiment 31, wherein the nucleic acids obtained comprise RNA, and wherein the RNA comprises mRNA, tRNA, rRNA, snRNA, or any combination thereof. 47. The method of embodiment 31, wherein the nucleic acids obtained comprise DNA. 48. The method of embodiment 31, wherein the supplement comprises a micronutrient. 49. The method of embodiment 48, wherein the micronutrient increases growth of a microbial taxon. 50. The method of embodiment 48, wherein the micronutrient increases a quantity of a microbial taxon. 51. The method of embodiment 48, wherein the micronutrient suppresses growth of a microbial taxon. 52. The method of embodiment 48, wherein the micronutrient decreases a quantity of a microbial taxon. 53. The method of embodiment 48, wherein the micronutrient increases a quantity of a first microbial taxon that competes with a second microbial taxon. 54. The method of embodiment 48, wherein the micronutrient increases a quantity of a first microbial taxon that increases a quantity of a second microbial taxon. 55. The method of embodiment 48, wherein the micronutrient increases the bioavailability of a substance that improves health. 56. The method of embodiment 48, wherein the micronutrient increases the uptake of a substance that improves health. 57. The method of embodiment 31, wherein the micronutrient comprises a mineral, a trace mineral, a vitamin, a biochemical substrate, or any combination thereof. 58. The method of embodiment 57, wherein the mineral is calcium, magnesium, sulfur, or any combination thereof. 59. The method of embodiment 57, wherein the trace mineral is iron, chromium, copper, fluoride, iodine, manganese, molybdenum, selenium, zinc, or any combination thereof. 60. The method of embodiment 57, wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof. 61. The method of embodiment 31, wherein the supplement comprises a prebiotic material, magnesium, fish oil, or any combination thereof.

62. A method of improving a health characteristic in a subject comprising: obtaining nucleic acids from a sample from the subject; obtaining sequence information representative of the nucleic acids; assigning a taxonomic designation to a portion of the nucleic acids having sequence signatures informative of the taxonomic designation; quantifying an amount of microorganisms in the sample corresponding to the taxonomic designation; comparing the quantity of microorganisms in the sample corresponding to the taxonomic designation to a reference value for the microorganism corresponding to the taxonomic designation; identifying a medication that alters the growth of microorganisms corresponding to the taxonomic designation; and recommending or administering to the subject the medication that alters the growth of microorganisms corresponding to the taxonomic designation. 63. The method of embodiment 62, wherein quantifying an amount of microorganisms comprises quantifying relative amounts of a plurality of microorganisms. 64. The method of embodiment 63, wherein administration of the medication adjusts the relative amounts of microorganisms in the subject to approach the reference value. 65. The method of embodiment 62, wherein the reference value represents a benchmark for the health characteristic. 66. The method of embodiment 62, wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. 67. The method of embodiment 66, wherein the sample comprises feces. 68. The method of embodiment 62, wherein the health characteristic comprises an immune system function, an endocrine system function, a digestive system function, a metabolic function, a mental health characteristic, or any combination thereof. 69. The method of embodiment 68, wherein the immune system function comprises an immune tolerance, an autoimmune disease, an inflammation, an infection, or any combination thereof. 70. The method of embodiment 69, wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. 71. The method of embodiment 69, wherein the infection comprises a respiratory infection, a urinary tract infection, a Clostridium difficile infection, or any combination thereof. 72. The method of embodiment 68, wherein the endocrine system function comprises a thyroid function, a hormone level, a cholesterol level change, or any combination thereof. 73. The method of embodiment 72, wherein the hormone level is a cortisol level, an estrogen level, an insulin level, a leptin level, a melatonin level, an oxytocin level, a testosterone level, or any combination thereof. 74. The method of embodiment 68, wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. 75. The method of embodiment 68, wherein the metabolic function comprises glucose metabolism, weight gain, weight loss, insulin level, fat mass accumulation, or any combination thereof. 76. The method of embodiment 68, wherein the mental health characteristic comprises anxiety, depression, Parkinson's disease, autism spectrum disorder, or a combination thereof. 77. The method of embodiment 62, wherein the nucleic acids obtained comprise RNA, and wherein the RNA comprises mRNA, tRNA, rRNA, snRNA, or any combination thereof. 78. The method of embodiment 62, wherein the nucleic acids obtained comprise DNA. 79. The method of embodiment 62, wherein the medication suppresses growth of microorganisms corresponding to the taxonomic designation. 80. The method of embodiment 79, wherein the medication is an antibiotic, an antiviral, an antipsychotic, an acid-reducing medication, a chemotherapy drug, or a blood-pressure medication. 81. The method of embodiment 80, wherein the antibiotic is selected from a penicillin, a cephalosporin, a macrolide, a fluoroquinolone, a sulfonamide, a tetracycline, or an aminoglycoside. 82. The method of embodiment 62, wherein the medication supports growth of microorganisms corresponding to the taxonomic designation.

83. A method of improving a health characteristic in a subject comprising: obtaining nucleic acids from a sample from the subject; obtaining sequence information representative of the nucleic acids; quantifying an amount of the nucleic acids in the sample corresponding to a biochemical activity; comparing the quantity of the biochemical activity in the sample to a reference value for the biochemical activity; identifying a nutrient that alters the amount of the biochemical activity in a gut of the subject; and recommending or administering to the subject a food containing the nutrient that alters the amount of the biochemical activity in the gut of the subject. 84. The method of embodiment 83, wherein administration of the food adjusts the biochemical activity in the subject to approach the reference value. 85. The method of embodiment 83, wherein the reference value represents a benchmark for the health characteristic. 86. The method of embodiment 83, wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. 87. The method of embodiment 86, wherein the sample comprises feces. 88. The method of embodiment 83, wherein the health characteristic comprises an immune system function, an endocrine system function, a digestive system function, a metabolic function, a mental health characteristic, or any combination thereof. 89. The method of embodiment 88, wherein the immune system function comprises an immune tolerance, an autoimmune disease, an inflammation, an infection, or any combination thereof. 90. The method of embodiment 89, wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. 91. The method of embodiment 89, wherein the infection comprises a respiratory infection, a urinary tract infection, a Clostridium difficile infection, or any combination thereof. 92. The method of embodiment 88, wherein the endocrine system function comprises a thyroid function, a hormone level, a cholesterol level change, or any combination thereof. 93. The method of embodiment 92, wherein the hormone level is a cortisol level, an estrogen level, an insulin level, a leptin level, a melatonin level, an oxytocin level, a testosterone level, or any combination thereof. 94. The method of embodiment 88, wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. 95. The method of embodiment 88, wherein the metabolic function comprises glucose metabolism, weight gain, weight loss, insulin level, fat mass accumulation, or any combination thereof. 96. The method of embodiment 88, wherein the mental health characteristic comprises anxiety, depression, Parkinson's disease, autism spectrum disorder, or a combination thereof. 97. The method of embodiment 83, wherein the nucleic acids obtained comprise RNA, and wherein the RNA comprises mRNA, tRNA, rRNA, snRNA, or any combination thereof. 98. The method of embodiment 83, wherein the nucleic acids obtained comprise DNA. 99. The method of embodiment 83, further comprising quantifying a small molecule informative of an amount of the biochemical activity. 100. The method of embodiment 99, wherein the quantifying comprises an enzymatic activity assay, a growth-inhibition culture, metabolic profiling, or any combination thereof. 101. The method of embodiment 99, wherein the small molecule comprises a metabolite generated by the biochemical activity. 102. The method of embodiment 99, wherein the small molecule comprises a short-chain fatty acid. 103. The method of embodiment 102, wherein the short-chain fatty acid comprises a butyrate. 104. The method of embodiment 99, wherein the small molecule comprises a propionate. 105. The method of embodiment 99, wherein the small molecule comprises a substrate of the biochemical activity. 106. The method of embodiment 83, wherein the food comprises a micronutrient. 107. The method of embodiment 106, wherein the micronutrient increases growth of a microbial taxon that generates the biochemical activity. 108. The method of embodiment 106, wherein the micronutrient increases a quantity of a microbial taxon that generates the biochemical activity. 109. The method of embodiment 106, wherein the micronutrient increases a level of the biochemical activity in the gut of the subject. 110. The method of embodiment 106, wherein the micronutrient suppresses growth of a microbial taxon that generates the biochemical activity. 111. The method of embodiment 106, wherein the micronutrient decreases a quantity of a microbial taxon that generates the biochemical activity. 112. The method of embodiment 106, wherein the micronutrient decreases the level of the biochemical activity in the gut of the subject. 113. The method of embodiment 106, wherein the micronutrient supplements the biochemical activity. 114. The method of embodiment 106, wherein the micronutrient activates the biochemical activity. 115. The method of embodiment 106, wherein the micronutrient inhibits the biochemical activity. 116. The method of embodiment 106, wherein the micronutrient increases a quantity of a first microbial taxon that competes with a second microbial taxon, wherein the second microbial taxon generates the biochemical activity. 117. The method of embodiment 106, wherein the micronutrient increases a quantity of a first microbial taxon that increases a quantity of a second microbial taxon, wherein the second microbial taxon generates the biochemical activity. 118. The method of embodiment 106, wherein the micronutrient increases the bioavailability of a substance that improves health. 119. The method of embodiment 106, wherein the micronutrient increases the uptake of a substance that improves health. 120. The method of embodiment 106, wherein the micronutrient comprises a mineral, a trace mineral, a vitamin, a biochemical substrate, or any combination thereof. 121. The method of embodiment 120, wherein the mineral is calcium, magnesium, sulfur, or any combination thereof. 122. The method of embodiment 120, wherein the trace mineral is iron, chromium, copper, fluoride, iodine, manganese, molybdenum, selenium, zinc, or any combination thereof 123. The method of embodiment 120, wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof. 124. The method of embodiment 83, further comprising: assigning a taxonomic designation to a portion of the nucleic acids having sequence signatures informative of the taxonomic designation; assigning the biochemical activity to the taxonomic designation; quantifying an amount of microorganisms in the sample corresponding to the taxonomic designation; and comparing the quantity of microorganisms in the sample corresponding to the taxonomic designation to a reference value for the microorganism corresponding to the taxonomic designation.

125. A method of improving a health characteristic in a subject comprising: obtaining nucleic acids from a sample from the subject; obtaining sequence information representative of the nucleic acids; quantifying an amount of the nucleic acids in the sample corresponding to a biochemical activity; comparing the quantity of the biochemical activity in the sample to a reference value for the biochemical activity; identifying a nutrient that alters the amount of the biochemical activity in a gut of the subject; and recommending or administering to the subject a supplement containing the nutrient that alters the amount of the biochemical activity in the gut of the subject. 126. The method of embodiment 125, wherein administration of the supplement adjusts the biochemical activity in the subject to approach the reference value. 127. The method of embodiment 125, wherein the reference value represents a benchmark for the health characteristic. 128. The method of embodiment 125, wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. 129. The method of embodiment 125, wherein the sample comprises feces. 130. The method of embodiment 125, wherein the health characteristic comprises an immune system function, an endocrine system function, a digestive system function, a metabolic function, a mental health characteristic, or any combination thereof. 131. he method of embodiment 130, wherein the immune system function comprises an immune tolerance, an autoimmune disease, an inflammation, an infection, or any combination thereof. 132. The method of embodiment 131, wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. 133. The method of embodiment 131, wherein the infection comprises a respiratory infection, a urinary tract infection, a Clostridium difficile infection, or any combination thereof. 134. The method of embodiment 130, wherein the endocrine system function comprises a thyroid function, a hormone level, a cholesterol level change, or any combination thereof. 135. The method of embodiment 134, wherein the hormone level is a cortisol level, an estrogen level, an insulin level, a leptin level, a melatonin level, an oxytocin level, a testosterone level, or any combination thereof. 136. The method of embodiment 130, wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. 137. The method of embodiment 130, wherein the metabolic function comprises glucose metabolism, weight gain, weight loss, insulin level, fat mass accumulation, or any combination thereof. 138. The method of embodiment 130, wherein the mental health characteristic comprises anxiety, depression, Parkinson's disease, autism spectrum disorder, or a combination thereof. 139. The method of embodiment 125, wherein the nucleic acids obtained comprise RNA, and wherein the RNA comprises mRNA, tRNA, rRNA, snRNA, or any combination thereof. 140. The method of embodiment 125, wherein the nucleic acids obtained comprise DNA. 141. The method of embodiment 125, further comprising quantifying a small molecule informative of an amount of the biochemical activity. 142. The method of embodiment 141, wherein the quantifying comprises an enzymatic activity assay, a growth-inhibition culture, metabolic profiling, or any combination thereof. 143. The method of embodiment 141, wherein the small molecule comprises a metabolite generated by the biochemical activity. 144. The method of embodiment 141, wherein the small molecule comprises a short-chain fatty acid. 145. The method of embodiment 144, wherein the short-chain fatty acid comprises a butyrate. 146. The method of embodiment 141, wherein the small molecule comprises a propionate. 147. The method of embodiment 141, wherein the small molecule comprises a substrate of the biochemical activity. 148. The method of embodiment 125, wherein the supplement comprises a micronutrient. 149. The method of embodiment 148, wherein the micronutrient increases growth of a microbial taxon that generates the biochemical activity. 150. The method of embodiment 148, wherein the micronutrient increases a quantity of a microbial taxon that generates the biochemical activity. 151. The method of embodiment 148, wherein the micronutrient increases a level of the biochemical activity in the gut of the subject. 152. The method of embodiment 148, wherein the micronutrient suppresses growth of a microbial taxon that generates the biochemical activity. 153. The method of embodiment 148, wherein the micronutrient decreases a quantity of a microbial taxon that generates the biochemical activity. 154. The method of embodiment 148, wherein the micronutrient decreases the level of the biochemical activity in the gut of the subject. 155. The method of embodiment 148, wherein the micronutrient supplements the biochemical activity. 156. The method of embodiment 148, wherein the micronutrient activates the biochemical activity. 157. The method of embodiment 148, wherein the micronutrient inhibits the biochemical activity. 158. The method of embodiment 148, wherein the micronutrient increases a quantity of a first microbial taxon that competes with a second microbial taxon, wherein the second microbial taxon generates the biochemical activity. 159. The method of embodiment 148, wherein the micronutrient increases a quantity of a first microbial taxon that increases a quantity of a second microbial taxon, wherein the second microbial taxon generates the biochemical activity. 160. The method of embodiment 148, wherein the micronutrient increases the bioavailability of a substance that improves health. 161. The method of embodiment 148, wherein the micronutrient increases the uptake of a substance that improves health. 161. The method of embodiment 125, wherein the supplement comprises a mineral, a trace mineral, a vitamin, a biochemical substrate, or any combination thereof. 163. The method of embodiment 162, wherein the mineral is calcium, magnesium, sulfur, or any combination thereof. 164. The method of embodiment 162, wherein the trace mineral is iron, chromium, copper, fluoride, iodine, manganese, molybdenum, selenium, zinc, or any combination thereof. 165. The method of embodiment 162, wherein the vitamin is thiamin (B1), riboflavin (B2), niacin, Vitamin B6, cobalamin (B12), folate, ascorbic acid, Vitamin A, Vitamin D, Vitamin E, Vitamin K, or any combination thereof. 166. The method of embodiment 125, wherein the supplement comprises a prebiotic material, magnesium, fish oil, or any combination thereof. 167. The method of embodiment 125, further comprising: assigning a taxonomic designation to a portion of the nucleic acids having sequence signatures informative of the taxonomic designation; assigning the biochemical activity to the taxonomic designation; quantifying an amount of microorganisms in the sample corresponding to the taxonomic designation; and comparing the quantity of microorganisms in the sample corresponding to the taxonomic designation to a reference value for the microorganism corresponding to the taxonomic designation.

168. A method of improving a health characteristic in a subject comprising: obtaining nucleic acids from a sample from the subject; obtaining sequence information representative of the nucleic acids; quantifying an amount of the nucleic acids in the sample corresponding to a biochemical activity; comparing the quantity of the biochemical activity in the sample to a reference value for the biochemical activity; identifying a nutrient that alters the amount of the biochemical activity in a gut of the subject; and recommending or administering to the subject a medication that alters the amount of the biochemical activity in the gut of the subject. 169. The method of embodiment 168, wherein administration of the medication adjusts the biochemical activity in the subject to approach the reference value. 170. The method of embodiment 168, wherein the reference value represents a benchmark for the health characteristic. 171. The method of embodiment 168, wherein the sample comprises earwax, sweat, breast milk, hair, blood, bile, cerebrospinal fluid, lymphatic fluid, semen, vaginal discharge, menstrual fluid, feces, sputum, urine, saliva, secretions from open wounds, secretions from the eye, skin tissue, a skin biopsy, subcutaneous tissue, muscle tissue, adipose tissue, or a combination thereof. 172. The method of embodiment 171, wherein the sample comprises feces. 173. The method of embodiment 168, wherein the health characteristic comprises an immune system function, an endocrine system function, a digestive system function, a metabolic function, a mental health characteristic, or any combination thereof. 174. The method of embodiment 173, wherein the immune system function comprises an immune tolerance, an autoimmune disease, an inflammation, an infection, or any combination thereof. 175. The method of embodiment 174, wherein the autoimmune disease comprises allergy, celiac disease, diabetes mellitus type 1, Graves' disease, inflammatory bowel disease, multiple sclerosis, psoriasis, rheumatoid arthritis, systemic lupus erythematosus, or any combination thereof. 176. The method of embodiment 174, wherein the infection comprises a respiratory infection, a urinary tract infection, a Clostridium difficile infection, or any combination thereof. 177. The method of embodiment 173, wherein the endocrine system function comprises a thyroid function, a hormone level, a cholesterol level change, or any combination thereof. 178. The method of embodiment 177, wherein the hormone level is a cortisol level, an estrogen level, an insulin level, a leptin level, a melatonin level, an oxytocin level, a testosterone level, or any combination thereof. 179. The method of embodiment 173, wherein the digestive system function comprises inflammatory bowel disease (IBD), ulcerative colitis, Crohn's disease, irritable bowel syndrome (IBS), necrotizing enterocolitis, colic, constipation, lactose intolerance, or any combination thereof. 180. The method of embodiment 173, wherein the metabolic function comprises glucose metabolism, weight gain, weight loss, insulin level, fat mass accumulation, or any combination thereof. 181. The method of embodiment 173, wherein the mental health characteristic comprises anxiety, depression, Parkinson's disease, autism spectrum disorder, or a combination thereof. 182. The method of embodiment 168, wherein the nucleic acids obtained comprise RNA, and wherein the RNA comprises mRNA, tRNA, rRNA, snRNA, or any combination thereof. 183. The method of embodiment 168, wherein the nucleic acids obtained comprise DNA. 184. The method of embodiment 168, further comprising quantifying a small molecule informative of an amount of the biochemical activity. 185. The method of embodiment 184, wherein the quantifying comprises an enzymatic activity assay, a growth-inhibition culture, metabolic profiling, or any combination thereof. 186. The method of embodiment 184, wherein the small molecule comprises a metabolite generated by the biochemical activity. 187. The method of embodiment 184, wherein the small molecule comprises a short-chain fatty acid. 188. The method of embodiment 187, wherein the short-chain fatty acid comprises a butyrate. 189. The method of embodiment 184, wherein the small molecule comprises a propionate. 190. The method of embodiment 184, wherein the small molecule comprises a substrate of the biochemical activity. 191. The method of embodiment 168, wherein the medication suppresses growth of microorganisms corresponding to the taxonomic designation. 192. The method of embodiment 191, wherein the medication is an antibiotic, an antiviral, an antipsychotic, an acid-reducing medication, a chemotherapy drug, or a blood-pressure medication. 193. The method of embodiment 192, wherein the antibiotic is selected from a penicillin, a cephalosporin, a macrolide, a fluoroquinolone, a sulfonamide, a tetracycline, or an aminoglycoside. 194. The method of embodiment 168, wherein the medication supports growth of microorganisms corresponding to the taxonomic designation. 195. The method of embodiment 168, further comprising: assigning a taxonomic designation to a portion of the nucleic acids having sequence signatures informative of the taxonomic designation; assigning the biochemical activity to the taxonomic designation; quantifying an amount of microorganisms in the sample corresponding to the taxonomic designation; and comparing the quantity of microorganisms in the sample corresponding to the taxonomic designation to a reference value for the microorganism corresponding to the taxonomic designation.

196. A method of taxonomically classifying organisms in a heterogeneous sample comprising: obtaining ribonucleic acids from a heterogeneous sample; obtaining sequence information representative of the ribonucleic acids; assaying for the presence of a plurality of sequence signatures in the sequence information informative of a taxonomic designation; categorizing the sample as comprising an organism corresponding to the taxonomic designation; and identifying a recommendation consistent with the presence of the organism corresponding to the taxonomic designation.

197. A method of taxonomically assessing the biochemical activity of a heterogeneous sample comprising: obtaining ribonucleic acids from a heterogeneous sample; obtaining sequence information representative of the ribonucleic acids; assigning the ribonucleic acids to open reading frames; identifying biochemical activities in the sample consistent with the open reading frames; and identifying a recommendation consistent with the presence of the biochemical activity.

198. A method of identifying a health recommendation for a subject comprising: obtaining ribonucleic acids from a heterogeneous sample; obtaining sequence information representative of the ribonucleic acids; assaying for a plurality of sequence signatures in the sample informative of a taxonomic designation; quantifying an amount of an organism corresponding to the taxonomic designation present in the sample; and identifying a health recommendation consistent with the amount of the organism corresponding to the taxonomic designation.

199. A method of taxonomically assessing the biochemical activity of a heterogeneous sample comprising: obtaining ribonucleic acids from a heterogeneous sample; obtaining sequence information representative of the ribonucleic acids; assigning taxonomic designations to a portion of the ribonucleic acids having sequence signatures informative of a taxonomic designation; assigning the ribonucleic acids to open reading frames consistent with the taxonomic designations; quantifying a biochemical activity in the sample consistent with the open reading frames; and identifying a recommendation consistent with the quantity of the biochemical activity.

200. A method of assessing gut shedding in a subject comprising: obtaining a nucleic acids from a stool sample; obtaining sequence information representative of the nucleic acids; assaying for a presence of a plurality of sequence signatures in the stool sample informative of a category of organisms; quantifying an amount of nucleic acid derived from the subject in the sample; quantifying an amount of nucleic acid derived from microorganisms in the sample; and assessing gut shedding in the subject by determining the proportion of nucleic acid derived from the subject to the proportion of nucleic acid derived from microorganisms. 201. The method of embodiment 200, further comprising generating a health recommendation based on the assessing gut shedding in the subject.

202. A method of identifying antibiotic resistant microorganisms in a heterogeneous microbial sample from a subject comprising: obtaining a ribonucleic acids from a heterogeneous microbial sample; obtaining sequence information representative of the ribonucleic acids; assaying for a presence of one or more sequence signatures in the heterogeneous microbial sample informative of a microorganism that is resistant to an antibiotic; and providing an effective amount of a different antibiotic to the subject to slow growth of the microorganism.

203. A method of treating a bacterial infection in a subject comprising: obtaining a ribonucleic acids from a heterogeneous microbial sample from the subject; obtaining sequence information representative of the ribonucleic acids; assaying for a presence of a plurality of sequence signatures in the heterogeneous microbial sample informative of a microorganism that is sensitive to an antibiotic; and providing an effective amount of the antibiotic to the subject to slow growth of the microorganism.

204. A method of assessing a metabolism in a subject comprising: assaying for nucleic acids corresponding to taxa in a gut microbiome of the subject that enhance metabolism; and assaying for nucleic acids corresponding to taxa in the gut microbiome of the subject that reduce metabolism.

205. A method of assessing metabolism in a subject comprising: assaying for nucleic acids corresponding to taxa in a gut microbiome of the subject that enhance metabolism; and assaying for nucleic acids corresponding to taxa in the gut microbiome of the subject that reduce metabolism; and assessing metabolic activity in the subject in the subject based on the taxa in a gut microbiome of the subject that enhance metabolism and the taxa in the gut microbiome of the subject that reduce metabolism in the gut microbiome. 206. The method of embodiment 205, further comprising administering to the subject a probiotic that modulates metabolism-relevant taxa in the gut microbiome. 207. The method of embodiment 205, further comprising recommending to the subject a diet that modulates metabolism-relevant taxa in the subject's gut.

208. A method comprising: a) generating a dataset about a subject by: i) determining data from a gut microbiome from a subject by: (1) obtaining nucleic acids from a sample comprising gut microbiome from a subject; (2) obtaining sequence information representative of the nucleic acids; iii) determining from the sequence information, (A) quantitative measures of each of a plurality of different microbial taxa and (B) quantitative measures of activity of each of a plurality of different genes; ii) determining phenotypic data about a plurality of different phenotypic traits of the subject; b) determining from the dataset a score set comprising a plurality of scores selected from: (i) a metabolic fitness score, (ii) an inflammatory activity score, (iii) a gut lining shedding score, (iv) a microbial richness score and (v) a microbial diversity score; c) recommending or administering to the subject one or more of a food to improve one or more of the scores. 209. The method of embodiment 208 wherein recommended foods are partial rank ordered into a plurality of groups from more desirable to less desirable to improve the one or more scores. 210. The method of embodiment 208 wherein the plurality of groups is 2, 3, 4 or 5.

211. A method comprising: a) generating a dataset about a subject by: i) determining data from a gut microbiome from a subject by: (1) obtaining nucleic acids from a sample comprising gut microbiome from a subject; (2) obtaining sequence information representative of the nucleic acids; iii) determining from the sequence information, (A) quantitative measures of each of a plurality of different microbial taxa and (B) quantitative measures of activity of each of a plurality of different genes; ii) determining phenotypic data about a plurality of different phenotypic traits of the subject; b) determining from the dataset a score set comprising a plurality of scores selected from: (i) a metabolic fitness score, (ii) an inflammatory activity score, (iii) a gut lining shedding score, (iv) a microbial richness score and (v) a microbial diversity score; c) recommending or administering to the subject one or more of a supplement to improve one or more of the scores. 212. The method of embodiment 211 wherein recommended supplements are partial rank ordered into a plurality of groups from more desirable to less desirable to improve the one or more scores. 213. The method of embodiment 211 wherein the plurality of groups is 2, 3, 4 or 5.

214. A method comprising: a) generating a dataset about a subject by: i) determining data from a gut microbiome from a subject by: (1) obtaining nucleic acids from a sample comprising gut microbiome from a subject; (2) obtaining sequence information representative of the nucleic acids; iii) determining from the sequence information, (A) quantitative measures of each of a plurality of different microbial taxa and (B) quantitative measures of activity of each of a plurality of different genes; ii) determining phenotypic data about a plurality of different phenotypic traits of the subject; b) determining from the dataset a score set comprising a plurality of scores selected from: (i) a metabolic fitness score, (ii) an inflammatory activity score, (iii) a gut lining shedding score, (iv) a microbial richness score and (v) a microbial diversity score; c) recommending or administering to the subject one or more of a medication to improve one or more of the scores. 215. The method of embodiment 214 wherein recommended medications are partial rank ordered into a plurality of groups from more desirable to less desirable to improve the one or more scores. 216. The method of embodiment 214 wherein the plurality of groups is 2, 3, 4 or 5.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising:
a) generating a dataset about a subject by:
i) determining data from a gut microbiome from a subject by:
(1) obtaining nucleic acids from a sample comprising gut microbiome from a subject;
(2) obtaining sequence information representative of the nucleic acids;
ii) determining from the sequence information, (A) quantitative measures of each of a plurality of different microbial taxa and (B) quantitative measures of activity of each of a plurality of different genes;
b) determining from the dataset a functional activity score for at least one functional category;
c) determining that the functional activity score is outside a reference range; and
d) recommending alterations in a diet for, or altering a diet of, the subject to alter the gut microbiome, wherein altering the gut microbiome shifts the functional score toward or into the reference range.

2. The method of claim 1, wherein the functional category includes one or more biochemical pathway components, one or more taxa group components or a combination of at least one biochemical pathway component and at least one taxa group component.

3. The method of claim 1, wherein the functional category is an aggregated functional category comprising a plurality of functional subcategories, each subcategory including at least one biochemical pathway component and at least one taxa group component.

4. The method of claim 1, wherein the quantitative measures of activity the plurality of genes are used to determine a quantitative measure of activity of a biochemical pathway.

5. The method of claim 1, wherein the quantitative measures of the plurality of microbial taxa are used to determine a quantitative measure of activity of a taxa group.

6. The method of claim 1, comprising, before operation (b):
determining from the dataset a biochemical pathway activity component and/or a taxa group activity component.

7. The method of claim 1, wherein the nucleic acids comprise ribonucleic acids.

8. The method of claim 2, wherein the ribonucleic acids comprise RNA.

9. The method of claim 2, wherein the ribonucleic acids comprise mRNA.

10. The method of claim 2, wherein the ribonucleic acids are essentially free of non-informative RNA.

11. The method of claim 1, wherein the quantitative measures are absolute measures or relative measures.

12. The method of claim 1, wherein at least one functional category is selected from the group consisting of inflammatory activity, metabolic fitness, digestive efficiency, intestinal barrier health, protein fermentation, gas production, microbial richness, butyrate production pathways, LPS biosynthesis pathways, methane gas production pathways, sulfide gas production pathways, flagellar assembly pathways, ammonia production pathways, putrescine production pathways, oxalate metabolism pathways, uric acid production pathways, salt stress pathways, biofilm chemotaxis and virulence pathways, TMA production pathways, primary bile acid pathways, secondary bile acid pathways, salt stress pathways, fucose metabolism pathways, cadaverine production pathways.

13. The method of claim 1, wherein a reference range is determined from a plurality of control subjects, wherein the plurality is at least any of 100 subjects, 1000 subjects, 5000, subjects or 10,000 subjects.

14. The method of claim 1, wherein an activity score outside of reference range is above or below the reference range.

15. The method of claim 1, wherein alterations in the diet comprise one or more of (1) adding to the diet microbes of one or more taxa for which there is a deficiency in the gut of the subject; (2) adding to the diet food or nutrients that promote growth of microbes of one or more taxa for which there is a deficiency; (3) adding to the diet a food or supplement comprising a nutrient that is deficient in the diet; and (4) adding to the diet a food or supplement comprising a nutrient that suppresses growth of microbes of one or more taxa for which there is an excess in the gut of the subject.

16. The method of claim 1, wherein recommending comprises transmitting recommendations to the subject over an electronic communications network.

17. The method of claim 16, wherein transmitting comprises posting recommendations to a website, e.g., a password-protected website, that is accessible to the subject.

18. A method comprising:
a) determining for a subject a functional activity score for at least one functional category, where in the functional activity score is derived from a dataset comprising nucleic acid sequence-derived quantitative measures of each of a plurality of different microbial taxa and quantitative measures of activity of each of a plurality of different genes;
b) determining that the functional activity score is outside a reference range; and
c) transmitting to the subject a dietary item determined, when included in the diet of the subject, to alter the gut microbiome so as to shift the functional score toward or into the reference range.

19. The method of claim 18, further comprising, before operation (a), receiving a gut microbiome sample from the subject and sequencing microbial nucleic acids in the sample to produce nucleic acid sequences.

20. A method comprising:
(a) receiving from the subject a sample comprising microbes from the subject's microbiome;
(b) sequencing a transcriptome from the microbiome;
(c) determining, from the sequence transcriptome, a functional utility score for each of one or more functional categories;
(d) determining that the functional utility score is outside a reference range;
(e) generating a dietary model including one or more dietary items which, when included in a diet of the subject, will move functional score toward or into the reference range; and
(f) providing the dietary model or at least one of the dietary items to the subject.

21. A method of improving a health characteristic in a subject comprising:
obtaining nucleic acids from a sample from the subject;
obtaining sequence information representative of the nucleic acids;
assigning a taxonomic designation to a portion of the nucleic acids having sequence signatures informative of the taxonomic designation;
quantifying an amount of microorganisms in the sample corresponding to the taxonomic designation;
comparing the quantity of microorganisms in the sample corresponding to the taxonomic designation to a reference value for the microorganism corresponding to the taxonomic designation;
identifying a nutrient or medication that alters the growth of microorganisms corresponding to the taxonomic designation; and
recommending or administering to the subject a food or supplement comprising the nutrient, or the medication that alters the growth of microorganisms corresponding to the taxonomic designation.

22. A method of improving a health characteristic in a subject comprising:
obtaining nucleic acids from a sample from the subject;
obtaining sequence information representative of the nucleic acids;
quantifying an amount of the nucleic acids in the sample corresponding to a biochemical (metabolic or signaling) pathway activity;
comparing the quantity of the biochemical activity in the sample to a reference value for the biochemical activity;
identifying a nutrient that alters the amount of the biochemical activity in a gut of the subject; and
recommending or administering to the subject a food or supplement containing the nutrient or medication that alters the amount of the biochemical activity in the gut of the subject.

23. A method of taxonomically assessing the biochemical activity of a heterogeneous sample comprising:
obtaining ribonucleic acids from a heterogeneous sample;
obtaining sequence information representative of the ribonucleic acids;
assigning the ribonucleic acids to open reading frames;
identifying biochemical activities in the sample consistent with the open reading frames; and
identifying a recommendation consistent with the presence of the biochemical activity.

24. A method of assessing gut shedding in a subject comprising:
obtaining nucleic acids from a stool sample;
obtaining sequence information representative of the nucleic acids;
assaying for a presence of a plurality of sequence signatures in the stool sample informative of a category of organisms;
quantifying an amount of nucleic acid derived from the subject in the sample;
quantifying an amount of nucleic acid derived from microorganisms in the sample; and
assessing gut shedding in the subject by determining the proportion of nucleic acid derived from the subject to the proportion of nucleic acid derived from microorganisms.

25. A method of identifying antibiotic resistant microorganisms in a heterogeneous microbial sample from a subject comprising:
obtaining ribonucleic acids from a heterogeneous microbial sample;
obtaining sequence information representative of the ribonucleic acids;
assaying for a presence of one or more sequence signatures in the heterogeneous microbial sample informative of a microorganism that is resistant to an antibiotic; and
providing an effective amount of a different antibiotic to the subject to slow growth of the microorganism.

26. A method of treating a bacterial infection in a subject comprising:
obtaining ribonucleic acids from a heterogeneous microbial sample from the subject;
obtaining sequence information representative of the ribonucleic acids;
assaying for a presence of a plurality of sequence signatures in the heterogeneous microbial sample informative of a microorganism that is sensitive to an antibiotic; and
providing an effective amount of the antibiotic to the subject to slow growth of the microorganism.

27. A method of assessing a metabolism in a subject comprising:
assaying for nucleic acids corresponding to taxa in a gut microbiome of the subject that enhance metabolism; and
assaying for nucleic acids corresponding to taxa in the gut microbiome of the subject that reduce metabolism.

28. A method of assessing metabolism in a subject comprising:
assaying for nucleic acids corresponding to taxa in a gut microbiome of the subject that enhance metabolism; and
assaying for nucleic acids corresponding to taxa in the gut microbiome of the subject that reduce metabolism; and
assessing metabolic activity in the subject in the subject based on the taxa in a gut microbiome of the subject that enhance metabolism and the taxa in the gut microbiome of the subject that reduce metabolism in the gut microbiome.
